(19) United States
(12) Patent Application Publication
(10) Pub. No.: US 2012/0077831 A1
(43) Pub. Date: Mar. 29, 2012

(54) METHODS AND ASSAYS FOR THE TREATMENT OF IRRITABLE BOWEL SYNDROME

(76) Inventor: Philip Manton Brown, Dallas, TX (US)

(21) Appl. No.: 13/192,571

(22) Filed: Jul. 28, 2011

Related U.S. Application Data
(63) Continuation-in-part of application No. 12/949,978, filed on Nov. 19, 2010.
(60) Provisional application No. 61/263,565, filed on Nov. 23, 2009.

(51) Int. Cl. A61K 31/513; A61P 1/00; G01N 33/98

(52) U.S. Cl. 514/272; 435/7.92

(57) ABSTRACT

Methods of treating irritable bowel syndrome (IBS) are disclosed. Assays and kits useful in the treatment of IBS are also disclosed.

This application is a continuation-in-part of U.S. patent application Ser. No. 12/949,978, filed Nov. 19, 2010, which claims priority to U.S. provisional patent application No. 61/263,565, filed Nov. 23, 2009, the entireties of which are incorporated herein by reference.

### 1. FIELD OF THE INVENTION

This invention relates to methods of treating irritable bowel syndrome, and assays and kits useful therein.

### 2. BACKGROUND

Irritable bowel syndrome (IBS) is the most common diagnosis made by gastroenterologists, and one of the most common disorders in the general population, with prevalence rates estimated at 10-15% of the U.S. population. IBS is a GI motility disorder that is characterized by abdominal pain or discomfort associated with a change in bowel habits such as constipation (IBS-C), diarrhea (IBS-D), or alternating between the two conditions (IBS-A). Mixed IBS (IBS-mixed or IBS-M) is used to define a subset of patients with IBS who at any one time exhibit symptoms of both diarrhea and constipation. This distinguishes IBS-M patients from IBS-A patients where the latter alternate between exhibiting features of IBS-D and IBS-C over periods of time.

Diagnostic criteria have been developed which aid the standardized approach to evaluating and diagnosing patients. Criteria that have been established over the previous three decades include: Manning criteria, Rome I, Rome II, and Rome III. See, e.g., Rome III: The Functional Gastrointestinal Disorders, Drossman, D. A., ed. (3^{rd} edition, January 2006), Appendix A; Drossman, D. A., Gastroenterology 20(5):1377-1390 (2006).

5-Hydroxytryptamine (5-HT) released from the enterochromaffin (EC) cells of the gastrointestinal (GI) tract is known to play a key role in the normal functioning of the gastrointestinal tract. Atkinson, W. et al., Gastroenterology 130:34-43, 34 (2006). 5-HT found in the blood is almost entirely derived from the EC cells of the GI tract. Id. It has been reported that some IBS-D patients have increased levels of blood 5-HT and its metabolite 5-hydroxyindoleacetic acid (5-HIAA) as compared to normal controls. See id.

### 3. SUMMARY OF THE INVENTION

This invention encompasses methods of treating and managing irritable bowel syndrome (IBS). For example, one embodiment of the invention encompasses a method of treating or managing IBS, which comprises administering to a patient suffering from IBS a therapeutically or prophylactically effective amount of a TPH inhibitor, wherein the patient suffering from IBS exhibits a baseline peripheral 5-HT level that is greater than the average peripheral 5-HT level exhibited by people who do not suffer from IBS.

Another embodiment encompasses a method of treating or managing IBS, which comprises administering to a patient suffering from IBS a therapeutically or prophylactically effective amount of a TPH inhibitor, wherein the patient suffering from IBS exhibits a baseline peripheral 5-HIAA level that is greater than the average peripheral 5-HIAA level exhibited by people who do not suffer from IBS.

This invention also encompasses methods and kits for determining whether an IBS patient is likely to respond to TPH inhibitor therapy. For example, one embodiment of the invention encompasses a method of determining if a patient suffering from IBS will be responsive to TPH inhibitor therapy. Another encompasses a kit for determining whether a patient suffering from IBS will be responsive to TPH inhibitor therapy.

### 4. BRIEF DESCRIPTION OF THE FIGURES

Aspects of the invention can be understood from the appended figures:

FIG. 1 shows the effect of the orally available TPH inhibitor (S)-2-amino-3-(4-(2-amino-6-((R)-2,2,2-trifluoro-1-(3′-methoxybiphenyl-4-yl)ethoxy)pyrimidin-4-yl)phenyl)propanoic acid on the urinary 5-HIAA levels of 40 normal, healthy volunteers in a phase 1b multi-dose clinical study.

FIG. 2 provides results obtained from a phase 2a study of the TPH inhibitor in non-constipating IBS patients. In particular, it shows that patients randomized to the high dose arm showed significant improvement versus placebo in weekly global assessment over the 28 day treatment period. The day the final dose was administered is indicated by the arrow.

FIG. 3 also provides results from the phase 2a study. In particular, patients randomized to the high dose arm showed significant improvement in stool consistency (Bristol Stool Scale).

FIG. 4 also provides results from the phase 2a study. In particular, a significant reduction in 24-hour urinary 5-HIAA upon treatment with the TPH inhibitor.

FIG. 5 shows the effect of the orally available TPH inhibitor (S)-2-amino-3-(4-(2-amino-6-((R)-1-(4-chloro-2-(3-methyl-1H-pyrazol-1-yl)phenyl)-2,2,2-trifluoroethoxy)-pyrimidin-4-yl)phenyl)propanoic acid on the mean urine 5-HIAA levels of normal healthy volunteers in a phase 1 clinical study. Volunteers received the compound or placebo for 14 days: the arrow indicates the last day of dosing.

FIG. 6 shows the compound's effect on the volunteers' mean plasma levels.

FIG. 7 shows the mean percent change from baseline plasma 5-HIAA levels upon dosing with the compound.

FIG. 8 shows the mean change from baseline plasma 5-HIAA levels upon dosing with the compound.

### 5. DETAILED DESCRIPTION

This invention is based, in part, on discoveries made during the first human clinical trials of a tryptophan hydroxylase (TPH) inhibitor for the treatment of IBS. When administered, TPH inhibitors lower the production of serotonin (5-HT) in the gastrointestinal tract.

### 5.1. Definitions

Unless otherwise indicated, the term “alkenyl” means a straight chain, branched and/or cyclic hydrocarbon having from 2 to 20 (e.g., 2 to 10 or 2 to 6) carbon atoms, and including at least one carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl and 3-decenyl.

Unless otherwise indicated, the term “alkoxy” means an —O-alkyl group. Examples of alkoxy groups include, but are not limited to, —OCH₃, —OCH₂CH₃, —O(CH₂)₂CH₃, —O(CH₂)₃CH₃, —O(CH₂)₄CH₃, and —O(CH₂)₅CH₃.

Unless otherwise indicated, the term “alkyl” means a straight chain, branched and/or cyclic (“cycloalkyl”) hydrocarbon having from 1 to 20 (e.g., 1 to 10 or 1 to 4) carbon atoms. Alkyl moieties having from 1 to 4 carbons are referred to as “lower alkyl.” Examples of alkyl groups include, but are not limited to, methyl, ethyl, propyl, isopropyl, n-butyl, t-butyl, isobutyl, pentyl, hexyl, isohexyl, heptyl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, cyclohexyl, and adamantyl. Additional examples of alkyl moieties have linear, branched and/or cyclic portions (e.g., 1-ethyl-4-methyl-cyclohexyl). The term “alkyl” includes saturated hydrocarbons as well as alkenyl and alkynyl moieties.

Unless otherwise indicated, the term “alkylaryl” or “alkyl-aryl” means an alkyl moiety bound to an aryl moiety.

Unless otherwise indicated, the term “alkylheteroaryl” or “alkyl-heteroaryl” means an alkyl moiety bound to a heteroaryl moiety.

Unless otherwise indicated, the term “alkylheterocycle” or “alkyl-heterocycle” means an alkyl moiety bound to a heterocycle moiety.

Unless otherwise indicated, the term “alkynyl” means a straight chain, branched or cyclic hydrocarbon having from 2 to 20 (e.g., 2 to 20 or 2 to 6) carbon atoms, and including at least one carbon-carbon triple bond. Representative alkynyl moieties include acetylenyl, propynyl, 1-butynyl, 2-butynyl, 1-pentynyl, 2-pentynyl, 3-methyl-1-butynyl, 4-pentynyl, 1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl and 9-decynyl.

Unless otherwise indicated, the term “aryl” means an aromatic ring or an aromatic or partially aromatic ring system composed of carbon and hydrogen atoms. An aryl moiety may comprise multiple rings bound or fused together. Examples of aryl moieties include, but are not limited to, anthracenyl, azulenyl, biphenyl, fluorenyl, indan, indenyl, naphthyl, phenanthrenyl, phenyl, 1,2,3,4-tetrahydro-naphthalene, and tolyl.

Unless otherwise indicated, the term “arylalkyl” or “aryl-alkyl” means an aryl moiety bound to an alkyl moiety.

Unless otherwise indicated, the terms “halogen” and “halo” encompass fluorine, chlorine, bromine, and iodine.

Unless otherwise indicated, the term “heteroalkyl” refers to an alkyl moiety (e.g., linear, branched or cyclic) in which at least one of its carbon atoms has been replaced with a heteroatom (e.g., N, O or S).

Unless otherwise indicated, the term “heteroalkylaryl” or “heteroalkyl-aryl” refers to a heteroalkyl moiety bound to an alkyl moiety.

Unless otherwise indicated, the term “heteroalkylheterocycle” or “heteroalkyl-heterocycle” refers to a heteroalkyl moiety bound to heterocycle moiety.

Unless otherwise indicated, the term “heteroaryl” means an aryl moiety wherein at least one of its carbon atoms has been replaced with a heteroatom (e.g., N, O or S). Examples include, but are not limited to, acridinyl, benzimidazolyl, benzofuranyl, benzoisothiazolyl, benzoisoxazolyl, benzoquinazolinyl, benzothiazolyl, benzoxazolyl, furyl, imidazolyl, indolyl, isothiazolyl, isoxazolyl, oxadiazolyl, oxazolyl, phthalazinyl, pyrazinyl, pyrazolyl, pyridazinyl, pyridyl, pyrimidinyl, pyrimidyl, pyrrolyl, quinazolinyl, quinolinyl, tetrazolyl, thiazolyl, and triazinyl.

Unless otherwise indicated, the term “heteroarylalkyl” or “heteroaryl-alkyl” means a heteroaryl moiety bound to an alkyl moiety.

Unless otherwise indicated, the term “heterocycle” refers to an aromatic, partially aromatic or non-aromatic monocyclic or polycyclic ring or ring system comprised of carbon, hydrogen and at least one heteroatom (e.g., N, O or S). A heterocycle may comprise multiple (i.e., two or more) rings fused or bound together. Heterocycles include heteroaryls. Examples include, but are not limited to, benzo[1,3]dioxolyl, 2,3-dihydro-benzo[1,4]dioxinyl, cinnolinyl, furanyl, hydantoinyl, morpholinyl, oxetanyl, oxiranyl, piperazinyl, piperidinyl, pyrrolidinonyl, pyrrolidinyl, tetrahydrofuranyl, tetrahydropyranyl, tetrahydropyridinyl, tetrahydropyrimidinyl, tetrahydrothiophenyl, tetrahydrothiopyranyl and valerolactamyl.

Unless otherwise indicated, the term “heterocyclealkyl” or “heterocycle-alkyl” refers to a heterocycle moiety bound to an alkyl moiety.

Unless otherwise indicated, the term “heterocycloalkyl” refers to a non-aromatic heterocycle.

Unless otherwise indicated, the term “heterocycloalkylalkyl” or “heterocycloalkyl-alkyl” refers to a heterocycloalkyl moiety bound to an alkyl moiety.

Unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

Unless otherwise indicated, the term “patient suffering from IBS” means a patient who exhibits symptoms of IBS (e.g., as defined in Rome III criteria). Symptoms of non-constipating IBS (i.e., IBS-D and/or IBS-A) include:
1. Recurrent abdominal pain or discomfort, defined as an uncomfortable sensation not described as pain at least 3 days/month and associated with two or more of the following characteristics:
   i. Improvement with defecation.
   ii. Onset associated with a change in frequency of stool.
   iii. Onset associated with a change in form (appearance) of stool.
2. Loose or watery stools for at least 25% of bowel movements and hard or lumpy stools for <25% of bowel movements (IBS-diarrhea), or loose or watery stools for at least 25% and hard or lumpy stool for at least 25% of bowel movements (IBS-mixed).

Unless otherwise indicated, the term “pharmaceutically acceptable salts” refers to salts prepared from pharmaceutically acceptable non-toxic acids or bases including inorganic acids and bases and organic acids and bases. Suitable pharmaceutically acceptable base addition salts include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Examples of specific salts thus include hydrochloride and mesylate salts. Others are well-known in the art. See, e.g., Remington's Pharmaceutical Sciences, 18th ed. (Mack Publishing, Easton Pa.: 1990) and Remington: The Science and Practice of Pharmacy, 19th ed. (Mack Publishing, Easton Pa.: 1995).

Unless otherwise indicated, a “prophylactically effective amount” of a compound is an amount sufficient to prevent a disease or condition, or one or more symptoms associated with the disease or condition, or prevent its recurrence. A “prophylactically effective amount” of a compound means an amount of therapeutic agent, alone or in combination with other agents, which provides a prophylactic benefit in the prevention of the disease. The term “prophylactically effective amount” can encompass an amount that improves overall prophylaxis or enhances the prophylactic efficacy of another prophylactic agent.

Unless otherwise indicated, the term “substituted,” when used to describe a chemical structure or moiety, refers to a derivative of that structure or moiety wherein one or more of its hydrogen atoms is substituted with a chemical moiety or functional group such as, but not limited to, alcohol, aldehyde, alkoxy, alkanoyloxy, alkoxycarbonyl, alkenyl, alkyl (e.g., methyl, ethyl, propyl, t-butyl), alkynyl, alkylcarbonyloxy (—OC(O)alkyl), amide (e.g. —C(O)NH-alkyl-, -alkylNHC(O)alkyl), amidinyl (e.g., —C(NH)NH-alkyl-, —C(NR)NH₂), amine (primary, secondary and tertiary such as alkylamino, arylamino, arylalkylamino), aroyl, aryl, aryloxy, azo, carbamoyl (e.g., —NHC(O)O-alkyl-, —OC(O)NH-alkyl), carbamyl (e.g., CONH₂, CONH-alkyl, CONH-aryl, CONH-arylalkyl), carbonyl, carboxyl, carboxylic acid, carboxylic acid anhydride, carboxylic acid chloride, cyano, ester, epoxide, ether (e.g., methoxy, ethoxy), guanidino, halo, haloalkyl (e.g., —CCl₃, —CF₃, —C(CF₃)₃), heteroalkyl, hemiacetal, imine (primary and secondary), isocyanate, isothiocyanate, ketone, nitrile, nitro, oxo, phosphodiester, sulfide, sulfonamido (e.g., SO₂NH₂), sulfone, sulfonyl (including alkylsulfonyl, arylsulfonyl and arylalkylsulfonyl), sulfoxide, thiol (e.g., sulfhydryl, thioether) and urea (e.g., —NHCONH-alkyl-).

Unless otherwise indicated, a “therapeutically effective amount” of a compound is an amount sufficient to provide a therapeutic benefit in the treatment or management of a disease or condition, or to delay or minimize one or more symptoms associated with the disease or condition. A “therapeutically effective amount” of a compound means an amount of therapeutic agent, alone or in combination with other therapies, which provides a therapeutic benefit in the treatment or management of the disease or condition. The term “therapeutically effective amount” can encompass an amount that improves overall therapy, reduces or avoids symptoms or causes of a disease or condition, or enhances the therapeutic efficacy of another therapeutic agent.

Unless otherwise indicated, the terms “treat,” “treating” and “treatment” contemplate an action that occurs while a patient is suffering from the specified disease or disorder, which reduces the severity of the disease or disorder, or retards or slows the progression of the disease or disorder.

Unless otherwise indicated, the term “include” has the same meaning as “include, but are not limited to,” and the term “includes” has the same meaning as “includes, but is not limited to.” Similarly, the term “such as” has the same meaning as the term “such as, but not limited to.”

Unless otherwise indicated, one or more adjectives immediately preceding a series of nouns is to be construed as applying to each of the nouns. For example, the phrase “optionally substituted alky, aryl, or heteroaryl” has the same meaning as “optionally substituted alky, optionally substituted aryl, or optionally substituted heteroaryl.”

It should be noted that a chemical moiety that forms part of a larger compound may be described herein using a name commonly accorded it when it exists as a single molecule or a name commonly accorded its radical. For example, the terms “pyridine” and “pyridyl” are accorded the same meaning when used to describe a moiety attached to other chemical moieties. Thus, the two phrases “XOH, wherein X is pyridyl” and “XOH, wherein X is pyridine” are accorded the same meaning, and encompass the compounds pyridin-2-ol, pyridin-3-ol and pyridin-4-ol.

It should also be noted that if the stereochemistry of a structure or a portion of a structure is not indicated with, for example, bold or dashed lines, the structure or the portion of the structure is to be interpreted as encompassing all stereoisomers of it. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

### 5.2. TPH Inhibitors

Compounds useful as TPH inhibitors include those disclosed in U.S. Pat. Nos. 7,723,345 and 7,553,840, both of which are incorporated herein.

Particular compounds are of the formula:

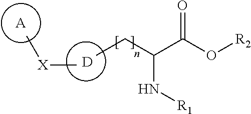

and pharmaceutically acceptable salts thereof, wherein: A is optionally substituted cycloalkyl, aryl, or heterocycle; X is a bond, —O—, —S—, —C(O)—, —C(R₄)═, ═C(R₄)—, —C(R₃R₄)—, —C(R₄)═C(R₄)—, —C≡C—, —N(R₅)—, —N(R₅)C(O)N(R₅)—, —C(R₃R₄)N(R₅)—, —N(R₅)C(R₃R₄)—, —ONC(R₃)—, —C(R₃)NO—, —C(R₃R₄)O—, —C(R₃R₄)O—, —OC(R₃R₄)—, —S(O₂)—, —S(O₂)N(R₅)—, —N(R₅)S(O₂)—, —C(R₃R₄)S(O₂)—, or —S(O₂)C(R₃R₄)—; D is optionally substituted aryl or heterocycle; R₁ is hydrogen or optionally substituted alkyl, alkyl-aryl, alkyl-heterocycle, aryl, or heterocycle; R2 is hydrogen or optionally substituted alkyl, alkyl-aryl, alkyl-heterocycle, aryl, or heterocycle; R3 is hydrogen, alkoxy, amino, cyano, halogen, hydroxyl, or optionally substituted alkyl; R₄ is hydrogen, alkoxy, amino, cyano, halogen, hydroxyl, or optionally substituted alkyl or aryl; each R₅ is independently hydrogen or optionally substituted alkyl or aryl; and n is 0-3.

Particular compounds are of the formula:

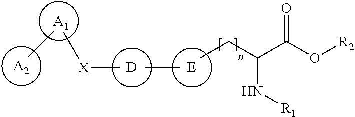

wherein: each of A₁ and A₂ is independently a monocyclic optionally substituted cycloalkyl, aryl, or heterocycle; and E is optionally substituted aryl or heterocycle. Some are of the formula:

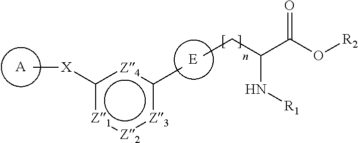

wherein: each of Z″₁, Z″₂, Z″₃, and Z″₄ is independently N or CR₁₀; each R₁₀ is independently amino, cyano, halogen, hydrogen, OR₁₁, SR₁₁, NR₁₂R₁₃, or optionally substituted alkyl, alkyl-aryl or alkyl-heterocycle; each R₁₁ is independently hydrogen or optionally substituted alkyl, alkyl-aryl or alkyl-heterocycle; each R₁₂ is independently hydrogen or optionally substituted alkyl, alkyl-aryl or alkyl-heterocycle; and each R₁₃ is independently hydrogen or optionally substituted alkyl, alkyl-aryl or alkyl-heterocycle.

Particular compounds are of the formula:

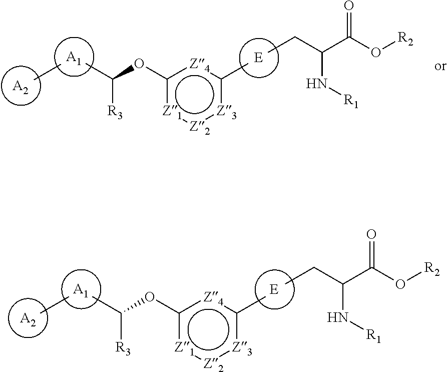

wherein, for example, R₃ is trifluoromethyl.

Some TPH inhibitors are compounds of the formula:

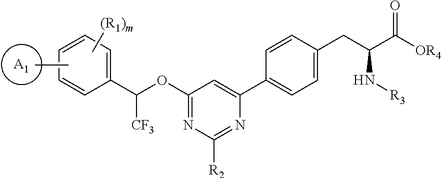

wherein: A₁ is optionally substituted heterocycle; each R₁ is independently halogen, hydrogen, C(O)R_{A}, OR_{A}, NR_{B}R_{C}, S(O₂)R_{A}, or optionally substituted alkyl, alkyl-aryl or alkyl-heterocycle; R₂ is independently halogen, hydrogen, C(O)R_{A}, OR_{A}, NR_{B}R_{C}, S(O₂)R_{A}, or optionally substituted alkyl, alkyl-aryl or alkyl-heterocycle; R₃ is hydrogen, C(O)R_{A}, C(O)OR_{A}, or optionally substituted alkyl, alkyl-aryl, alkyl-heterocycle, aryl, or heterocycle; R₄ is hydrogen or optionally substituted alkyl, alkyl-aryl, alkyl-heterocycle, aryl, or heterocycle; each R_{A} is independently hydrogen or optionally substituted alkyl, alkyl-aryl or alkyl-heterocycle; each R_{B} is independently hydrogen or optionally substituted alkyl, alkyl-aryl or alkyl-heterocycle; each R_{C} is independently hydrogen or optionally substituted alkyl, alkyl-aryl or alkyl-heterocycle; and m is 1-4.

Particular compounds are of the formula:

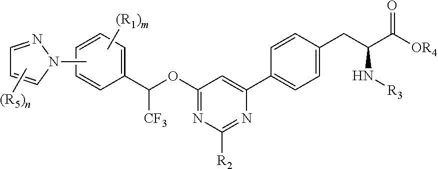

wherein: each R5 is independently halogen, hydrogen, C(O)R_{A}, OR_{A}, NR_{B}R_{C}, S(O₂)R_{A}, or optionally substituted alkyl, alkyl-aryl or alkyl-heterocycle; and n is 1-3. Some are of the formula:

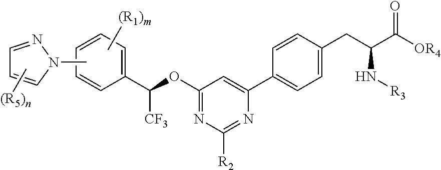

Specific TPH inhibitors include:

(S)-2-Amino-3-(4-(4-amino-6-((R)-1-(naphthalen-2-yl)ethylamino)-1,3,5-triazin-2-yl)phenyl)propanoic acid;

(S)-2-Amino-3-(4-(4-amino-6-((4′-methylbiphenyl-4-yl)methylamino)-1,3,5-triazin-2-yl)phenyl)propanoic acid;

(S)-2-Amino-3-(4-(4-morpholino-6-(naphthalen-2-ylmethylamino)-1,3,5-triazin-2-yl)phenyl)propanoic acid;

(2S)-2-Amino-3-(4-(2-amino-6-(2,2,2-trifluoro-1-(2-(trifluoromethyl)phenyl)ethoxy)pyrimidin-4-yl)phenyl)propanoic acid;

(2S)-2-Amino-3-(4-(2-amino-6-(2,2,2-trifluoro-1-p-tolylethoxy)pyrimidin-4-yl)phenyl)propanoic acid;

(2S)-2-Amino-3-(4-(2-amino-6-(1-cyclohexyl-2,2,2-trifluoroethoxy)pyrimidin-4-yl)phenyl)propanoic acid;

(S)-2-Amino-3-(4-(6-(2-fluorophenoxy)pyrimidin-4-yl)phenyl)propanoic acid;

(2S)-2-Amino-3-(4-(4-(3-(4-chlorophenyl)piperidin-1-yl)-1,3,5-triazin-2-yl)phenyl)propanoic acid;

(2S)-2-Amino-3-(4-(4-amino-6-(2,2,2-trifluoro-1-phenylethoxy)-1,3,5-triazin-2-yl)phenyl)propanoic acid;

(S)-2-Amino-3-(5-(4-amino-6-((R)-1-(naphthalen-2-yl)ethylamino)-1,3,5-triazin-2-yl)pyridin-2-yl)propanoic acid;

(S)-2-Amino-3-(3-(4-amino-6-((R)-1-(naphthalen-2-yl)ethylamino)-1,3,5-triazin-2-yl)-1H-pyrazol-1-yl)propanoic acid;

(S)-2-Amino-3-(4′-(3-(cyclopentyloxy)-4-methoxybenzylamino)biphenyl-4-yl)propanoic acid;

(S)-2-Amino-3-(4-(6-(3-(cyclopentyloxy)-4-methoxybenzylamino)pyrimidin-4-yl)phenyl)propanoic acid;

(S)-2-Amino-3-(4-(6-(3-(cyclopentyloxy)-4-methoxybenzylamino)pyrazin-2-yl)phenyl)propanoic acid;

(S)-2-Amino-3-(4-(5-((4′-methylbiphenyl-2-yl)methylamino)pyrazin-2-yl)phenyl)propanoic acid;

(2S)-2-Amino-3-(4-(6-(2,2,2-trifluoro-1-phenylethoxy)-pyrimidin-4-yl)phenyl)propanoic acid;

(2S)-2-Amino-3-(4-(6-(1-(3,4-difluorophenyl)-2,2,2-trifluoroethoxy)pyrimidin-4-yl)phenyl)propanoic acid;

(S)-2-Amino-3-(4-(5-(3-(cyclopentyloxy)-4-methoxybenzylamino)-pyrazin-2-yl)phenyl)propanoic acid;

(S)-2-Amino-3-(4-(5-((3-(cyclopentyloxy)-4-methoxybenzyl)-(methyl)amino)pyrazin-2-yl)phenyl)propanoic acid;

(S)-2-Amino-3-(4-(5-((1,3-dimethyl-1H-pyrazol-4-yl)methylamino)pyrazin-2-yl)phenyl)propanoic acid;

(S)-2-Amino-3-(4-(4-amino-6-((S)-1-(naphthalen-2-yl)ethylamino)-1,3,5-triazin-2-yloxy)phenyl)propanoic acid;

(S)-2-Amino-3-(4-(4-amino-6-((R)-1-(biphenyl-2-yl)-2,2,2-trifluoroethoxy)-1,3,5-triazin-2-yl)phenyl)propanoic acid;

(2S)-2-Amino-3-(4-(4-amino-6-(1-(6,8-difluoronaphthalen-2-yl)ethylamino)-1,3,5-triazin-2-yl)phenyl)propanoic acid;

(2S)-2-Amino-3-(4-(4-amino-6-(2,2,2-trifluoro-1-(3′-methylbiphenyl-2-yl)ethoxy)-1,3,5-triazin-2-yl)phenyl)propanoic acid;

(S)-2-Amino-3-(4-(5-(3,4-dimethoxyphenylcarbamoyl)-pyrazin-2-yl)phenyl)propanoic acid;

(S)-2-Amino-3-(4-(2-amino-6-(4-(2-(trifluoromethyl)phenyl)-piperidin-1-yl)pyrimidin-4-yl)phenyl)propanoic acid;

(S)-2-Amino-3-(4-(2-amino-6-((R)-1-(naphthalen-2-yl)ethylamino)pyrimidin-4-yl)phenyl)propanoic acid;

(S)-2-Amino-3-(4-(2-amino-6-(methyl((R)-1-(naphthalen-2-yl)ethyl)amino)pyrimidin-4-yl)phenyl)propanoic acid;

(S)-2-Amino-3-(4-(2-amino-6-((S)-2,2,2-trifluoro-1-(6-methoxynaphthalen-2-yl)ethoxy)pyrimidin-4-yl)phenyl)propanoic acid;

(S)-2-Amino-3-(4-(5-(biphenyl-4-ylmethylamino)pyrazin-2-yl)phenyl)propanoic acid;

(S)-2-Amino-3-(4-(5-(naphthalen-2-ylmethylamino)pyrazin-2-yl)phenyl)propanoic acid;

(S)-2-(Tert-butoxycarbonylamino)-3-(4-(5-(naphthalen-2-ylmethylamino)pyrazin-2-yl)phenyl)propanoic acid;

(S)-2-Morpholinoethyl 2-amino-3-(4-(5-(naphthalen-2-ylmethylamino)pyrazin-2-yl)phenyl)propanoate;

(2S)-2-Amino-3-(4-(2-amino-6-(2,2,2-trifluoro-1-(3′-fluorobiphenyl-4-yl)ethoxy)pyrimidin-4-yl)phenyl)propanoic acid;

(S)-2-Amino-3-(4-(2-amino-6-(benzylthio)pyrimidin-4-yl)phenyl)propanoic acid;

(S)-2-Amino-3-(4-(2-amino-6-(naphthalen-2-ylmethylthio)pyrimidin-4-yl)phenyl)propanoic acid;

(2S)-2-Amino-3-(4-(2-amino-6-(1-(3,4-difluorophenyl)-2,2,2-trifluoroethoxy)pyrimidin-4-yl)phenyl)propanoic acid;

(2S)-2-Amino-3-(4-(2-amino-6-(2,2,2-trifluoro-1-(3′-methylbiphenyl-2-yl)ethoxy)pyrimidin-4-yl)phenyl)propanoic acid;

(S)-2-Amino-3-(4-(5-(3-(cyclopentyloxy)-4-methoxybenzylamino)pyridin-3-yl)phenyl)propanoic acid;

2-Amino-3-(3-(4-amino-6-((R)-1-(naphthalen-2-yl)ethylamino)-1,3,5-triazin-2-yl)phenyl)propanoic acid;

2-Amino-3-(4-(4-amino-6-((R)-1-(naphthalen-2-yl)ethylamino)-1,3,5-triazin-2-yl)-2-fluorophenyl)propanoic acid;

(2S)-2-Amino-3-(4-(4-amino-6-(1-(adamantyll)ethylamino)-1,3,5-triazin-2-yl)phenyl)propanoic acid;

(S)-2-Amino-3-(4-(5-fluoro-4-((R)-1-(naphthalen-2-yl)ethylamino)pyrimidin-2-yl)phenyl)propanoic acid;

(S)-2-Amino-3-(4-(2-amino-6-(4-(trifluoromethyl)-benzylamino)pyrimidin-4-yl)phenyl)propanoic acid;

2-amino-3-(5-(5-phenylthiophen-2-yl)-1H-indol-3-yl)propanoic acid;

(S)-2-amino-3-(4-(4-(4-phenoxyphenyl)-1H-1,2,3-triazol-1-yl)phenyl)propanoic acid;

(S)-2-amino-3-(4-(4-(4-(thiophene-2-carboxamido)phenyl)-1H-1,2,3-triazol-1-yl)phenyl)propanoic acid;

(S)-2-amino-3-(4-(5-(4-(thiophene-2-carboxamido)phenyl)-1H-1,2,3-triazol-1-yl)phenyl)propanoic acid;

(S)-2-amino-3-(4-(2-amino-6-(phenylethynyl)pyrimidin-4-yl)phenyl)propanoic acid;

(S)-2-Amino-3-(4-(5-(2-fluoro-4,5-dimethoxybenzylamino)pyrazin-2-yl)phenyl)propanoic acid;

(S)-2-Amino-3-(4-(2-amino-6-(4-(2-methoxyphenyl)piperidin-1-yl)pyrimidin-4-yl)phenyl)propanoic acid;

(S)-2-Amino-3-(4-(6-(3-(cyclopentyloxy)-4-methoxybenzylamino)-2-(dimethylamino)pyrimidin-4-yl)phenyl)propanoic acid;

(S)-2-Amino-3-(4-(5-(3,4-dimethylbenzylamino)pyrazin-2-yl)phenyl)propanoic acid;

(S)-2-Amino-3-(4-(5-(biphenyl-2-ylmethylamino)pyrazin-2-yl)phenyl)propanoic acid;

(S)-Ethyl 2-amino-3-(4-(2-amino-6-(4-(trifluoromethyl)benzylamino)pyrimidin-4-yl)phenyl)propanoate;

(S)-2-Amino-3-(4-(5-(cyclopentylmethylamino)pyrazin-2-yl)phenyl)propanoic acid;

(2S)-2-Amino-3-(4-(2-amino-6-(3-(2-(trifluoromethyl)phenyl)pyrrolidin-1-yl)pyrimidin-4-yl)phenyl)propanoic acid;

(2S)-2-Amino-3-(4-(2-amino-6-(1,2,3,4-tetrahydronaphthalen-1-ylamino)pyrimidin-4-yl)phenyl)propanoic acid;

(S)-2-Amino-3-(4-(2-amino-6-((R)-1-(naphthalen-2-yl)ethoxy)pyrimidin-4-yl)phenyl)propanoic acid;

(2S)-2-Amino-3-(4-(2-amino-6-(1,2-diphenylethylamino)pyrimidin-4-yl)phenyl)propanoic acid;

(S)-2-Amino-3-(4-(2-amino-6-((R)-1-(4-(benzo[b]thiophen-3-yl)phenyl)ethylamino)pyrimidin-4-yl)phenyl)propanoic acid;

(S)-2-Amino-3-(4-(4-amino-6-((R)-1-(4′-methoxybiphenyl-4-yl)ethylamino)-1,3,5-triazin-2-yl)phenyl)propanoic acid;

2-Amino-3-(1-(4-amino-6-((R)-1-(naphthalen-2-yl)ethylamino)-1,3,5-triazin-2-yl)piperidin-4-yl)propanoic acid;

(2S)-2-Amino-3-(4-(4-amino-6-(1-(4-fluoronaphthalen-1-yl)ethylamino)-1,3,5-triazin-2-yl)phenyl)propanoic acid;

(S)-2-Amino-3-(4-(4-amino-6-((3′-fluorobiphenyl-4-yl)methylamino)-1,3,5-triazin-2-yl)phenyl)propanoic acid;

2-Amino-3-(4-(4-amino-6-((R)-1-(naphthalen-2-yl)ethylamino)-1,3,5-triazin-2-yl)-2-fluorophenyl)propanoic acid;

(S)-2-Amino-3-(4-(2-amino-6-((R)-2,2,2-trifluoro-1-(3′-methoxybiphenyl-4-yl)ethoxy)pyrimidin-4-yl)phenyl)propanoic acid;

(2S)-2-Amino-3-(4-(4-amino-6-(2,2,2-trifluoro-1-(3′-fluorobiphenyl-2-yl)ethoxy)-1,3,5-triazin-2-yl)phenyl)propanoic acid;

(2S)-2-Amino-3-(4-(4-amino-6-(1-(tert-butylphenyl)ethylamino)-1,3,5-triazin-2-yl)phenyl)propanoic acid;

(2S)-2-Amino-3-(4-(2-amino-6-(2,2,2-trifluoro-1-(3′-fluorobiphenyl-4-yl)ethoxy)pyrimidin-4-yl)phenyl)propanoic acid;

(2S)-2-Amino-3-(4-(4-amino-6-(6,7-dihydroxy-1-methyl-3,4-dihydroisoquinolin-2(1H)-yl)-1,3,5,-triazin-2-yl)phenylpropanoic acid;

(2S)-2-Amino-3-(4-(4-amino-6-(2,2,2-trifluoro-1-(3′-methylbiphenyl-4-yl)ethoxy)-1,3,5-triazin-2-yl)phenyl)propanoic acid;

(S)-2-Amino-3-(4-(4-amino-6-((R)-1-(naphthalen-2-yl)ethylamino)pyrimidin-2-yl)phenyl)propanoic acid;

(2S)-2-Amino-3-(4-(2-amino-6-(2,2,2-trifluoro-1-(4′-fluorobiphenyl-4yl)ethoxy)pyrimidin-4-yl)phenyl)propanoic acid;

(2S)-2-Amino-3-(4-(6-(3-(4-chlorophenoxy)piperidin-1-yl)phenyl)propanoic acid;

(S)-3-(4-(4-Amino-6-((R)-1-(naphthalen-2-yl)ethylamino)-1,3,5-triazin-2-yl)phenyl)-2-(2-aminoacetamido)propanoic acid;

(S)-2-Amino-3-(4-(6-((R)-1-(naphthalen-2-yl)ethylamino)-2-(trifluoromethyl)pyrimidin-4-yl)phenyl)propanoic acid;

(S)-2-Amino-3-(4-(2-amino-6-(4-(3-chlorophenyl)piperazin-1yl)pyrimidin-4-yl)pyrimidin-4-yl)phenyl)propanoic acid;

(S)-2-Amino-3-(4-(2-amino-6-((R)-2,2,2-trifluoro-1-phenylethoxy)pyrimidin-4-yl)phenyl)propanoic acid;

(2S)-2-Amino-3-(4-(2-amino-6-(1,4-diphenylbutylamino)pyrimidin-4-yl)phenyl)propanoic acid;

(2S)-2-Amino-3-(4-(6-(1-(3′-chlorobiphenyl-2-yl)-2,2,2-trifluoroethoxy)pyrimidin-4-yl)phenyl)propanoic acid;

(2S)-2-Amino-3-(4-(4-amino-6-(1-(biphenyl-4-yl)-2,2,2-trifluoroethoxy)-1,3,5-triazin-2-yl)phenyl)propanoic acid;

(2S)-2-Amino-3-(4-(2-amino-6-(2,2,3,3,3-pentafluoro-1-(3-fluoro-4-methylphenyl)propoxy)pyrimidin-4-yl)phenyl)propanoic acid;

(S)-Ethyl 2-amino-3-(4-(2-amino-6-((R)-2,2,2-trifluoro-1-(3′-methoxybiphenyl-4-yl)ethoxy)pyrimidin-4-yl)phenyl)propanoate;

(S)-2-Amino-3-(4-(2-amino-6-((S)-2,2,2-trifluoro-1-(3′-methoxybiphenyl-4-yl)ethoxy)pyrimidin-4-yl)phenyl)propanoic acid;

(2S)-2-Amino-3-(4-(2-amino-6-(2,2,2-trifluoro-1-(3-fluoro-3′-methoxybiphenyl-4-yl)ethoxy)pyrimidin-4-yl)phenyl)propanoic acid;

(2S)-2-Amino-3-(4-(2-amino-6-(1-(3′-(dimethylamino)biphenyl-2-yl)-2,2,2-trifluoroethoxy)pyrimidin-4-yl)phenyl)propanoic acid;

(2S)-2-Amino-3-(4-(2-amino-6-(2,2,2-trifluoro-1-(3′-methoxy-5-methylbiphenyl-2-yl)ethoxy)pyrimidin-4-yl)phenyl)propanoic acid;

(2S)-2-Amino-3-(4-(2-amino-6-(2,2,2-trifluoro-1-(4′-methoxy-5-methylbiphenyl-2-yl)ethoxy)pyrimidin-4-yl)phenyl)propanoic acid;

(2S)-2-Amino-3-(4-(2-amino-6-(2,2,2-trifluoro-1-(3′-methoxy-3-(methylsulfonyl)biphenyl-4-yl)ethoxy)pyrimidin-4-yl)phenyl)propanoic acid;

(2S)-2-Amino-3-(4-(2-amino-6-(1-(2-(cyclopropylmethoxy)-4-fluorophenyl)-2,2,2-trifluoroethoxy)pyrimidin-4-yl)phenyl)propanoic acid;

(2S)-2-Amino-3-(4-(6-(1-(2-(cyclopropylmethoxy)-4-fluorophenyl)-2,2,2-trifluoroethoxy)pyrimidin-4-yl)phenyl)propanoic acid;

(2S)-2-Amino-3-(4-(2-amino-6-(2,2,2-trifluoro-1-(2-(isopentyloxy)phenyl)ethoxy)pyrimidin-4-yl)phenyl)propanoic acid;

(2S)-2-Amino-3-(4-(5-(2,2,2-trifluoro-1-(3′-fluorobiphenyl-4-yl)ethoxy)pyrazin-2-yl)phenyl)propanoic acid;

(2S)-2-Amino-3-(4-(2-amino-6-(2,2,2-trifluoro-1-(4′-methoxybiphenyl-2-yl)ethoxy)pyrimidin-4-yl)phenyl)propanoic acid;

(2S)-2-Amino-3-(4-(2-amino-6-(1-(3′-carbamoylbiphenyl-2-yl)-2,2,2-trifluoroethoxy)pyrimidin-4-yl)phenyl)propanoic acid;

(2S)-2-Amino-3-(4-(2-amino-6-(1-(4′-carbamoylbiphenyl-2-yl)-2,2,2-trifluoroethoxy)pyrimidin-4-yl)phenyl)propanoic acid;

(2S)-2-Amino-3-(4-(2-amino-6-(2,2,2-trifluoro-1-(4-(2-methoxyphenoxy)phenyl)ethoxy)pyrimidin-4-yl)phenyl)propanoic acid;

(2S)-2-Amino-3-(4-(6-(2,2,2-trifluoro-1-(4-(2-methoxyphenoxy)phenyl)ethoxy)pyrimidin-4-yl)phenyl)propanoic acid;

(2S)-2-Amino-3-(4-(6-(2,2,2-trifluoro-1-(2-(isopentyloxy)phenyl)ethoxy)pyrimidin-4-yl)phenyl)propanoic acid;

(2S)-3-(4-(6-(1-(3′-Acetamidobiphenyl-2-yl)-2,2,2-trifluoroethoxy)-2-aminopyrimidin-4-yl)phenyl)-2-aminopropanoic acid;

(2S)-3-(4-(6-(1-(4′-Acetamidobiphenyl-2-yl)-2,2,2-trifluoroethoxy)-2-aminopyrimidin-4-yl)phenyl)-2-aminopropanoic acid;

(2S)-2-Amino-3-(4-(2-amino-6-(1-(4-cyanophenyl)-2,2,2-trifluoroethoxy)pyrimidin-4-yl)phenyl)propanoic acid;

(S)-Ethyl 2-amino-3-(4-(2-amino-6-((R)-2,2,2-trifluoro-1-p-tolylethoxy)pyrimidin-4-yl)phenyl)propanoate;

(2S)-2-Amino-3-(4-(2-amino-6-(2,2,2-trifluoro-1-(1-methoxybicyclo[2.2.2]oct-5-en-2-yl)ethoxy)pyrimidin-4-yl)phenyl)propanoic acid;

(2S)-2-Amino-3-(4-(2-amino-6-(1-(4-(cyclopentyloxy)phenyl)-2,2,2-trifluoroethoxy)pyrimidin-4-yl)phenyl)propanoic acid;

(2S)-2-Amino-3-(4-(6-(1-(4-(cyclopentyloxy)phenyl)-2,2,2-trifluoroethoxy)pyrimidin-4-yl)phenyl)propanoic acid;

(2S)-2-Amino-3-(4-(2-amino-6-(2,2,2-trifluoro-1-(4-(3-methoxyphenoxy)phenyl)ethoxy)pyrimidin-4-yl)phenyl)propanoic acid;

(2S)-2-Amino-3-(4-(2-amino-6-(1-(4,5-dimethoxybiphenyl-2-yl)-2,2,2-trifluoroethoxy)pyrimidin-4-yl)phenyl)propanoic acid;

(2S)-2-Amino-3-(4-(2-amino-6-(1-(4,5-dimethoxy-3′-methylbiphenyl-2-yl)-2,2,2-trifluoroethoxy)pyrimidin-4-yl)phenyl)propanoic acid;

(2S)-2-Amino-3-(4-(5-(2,2,2-trifluoro-1-(2′-methylbiphenyl-2-ypethoxy)pyrazin-2-yl)phenyl)propanoic acid; (2S)-2-Amino-3-(4-(6-(2,2,2-trifluoro-1-(4-(3-methoxyphenoxy)phenyl)ethoxy)pyrimidin-4-yl)phenyl)propanoic acid;

(2S)-2-Amino-3-(4-(2-amino-6-(1-(2-(3,5-difluorophenoxy)phenyl)-2,2,2-trifluoroethoxy)pyrimidin-4-yl)phenyl)propanoic acid;

(2S)-2-Amino-3-(4-(2-amino-6-(2,2,2-trifluoro-1-(4-(4-methoxyphenoxy)phenyl)ethoxy)pyrimidin-4-yl)phenyl)propanoic acid;

(2S)-2-Amino-3-(4-(2-amino-6-(1-(4′-((S)-2-amino-2-carboxyethyl)biphenyl-2-yl)-2,2,2-trifluoroethoxy)pyrimidin-4-yl)phenyl)propanoic acid;

(2S)-2-Amino-3-(4-(2-amino-6-(1-(2-bromophenyl)-2,2,2-trifluoroethoxy)pyrimidin-4-yl)phenyl)propanoic acid;

(2S)-2-Amino-3-(4-(5-(2,2,2-trifluoro-1-(3′-methylbiphenyl-2-yl)ethoxy)pyrazin-2-yl)phenyl)propanoic acid;

(2S)-2-Amino-3-(4-(2-amino-6-(2,2,2-trifluoro-1-(4-methoxybiphenyl-2-yl)ethoxy)pyrimidin-4-yl)phenyl)propanoic acid;

(2S)-2-Amino-3-(4-(5-(2,2,2-trifluoro-1-(2-(4-methylthiophen-3-yl)phenyl)ethoxy)pyrazin-2-yl)phenyl)propanoic acid;

(2S)-2-Amino-3-(4-(2-amino-6-(2,2,2-trifluoro-1-(4-methoxy-3′-methylbiphenyl-2-yl)ethoxy)pyrimidin-4-yl)phenyl)propanoic acid;

(2S)-2-Amino-3-(4-(2-amino-6-(2,2,2-trifluoro-1-(3′-(hydroxymethyl)biphenyl-2-yl)ethoxy)pyrimidin-4-yl)phenyl)propanoic acid;

(2S)-2-Amino-3-(4-(2-amino-6-(1-(3′-cyanobiphenyl-2-yl)-2,2,2-trifluoroethoxy)pyrimidin-4-yl)phenyl)propanoic acid;

(2S)-2-Amino-3-(4-(6-(1-(2-(3,5-difluorophenoxy)phenyl)-2,2,2-trifluoroethoxy)pyrimidin-4-yl)phenyl)propanoic acid;

(2S)-2-Amino-3-(4-(6-(2,2,2-trifluoro-1-(4-(4-methoxyphenoxy)phenyl)ethoxy)pyrimidin-4-yl)phenyl)propanoic acid;

(2S)-2-Amino-3-(4-(2-amino-6-(2,2,2-trifluoro-1-(2-(4-methylthiazol-2-yl)thiophen-3-yl)ethoxy)pyrimidin-4-yl)phenyl)propanoic acid;

(2S)-2-Amino-3-(4-(2-amino-6-(2,2,2-trifluoro-1-(5-(4-methoxyphenypisoxazol-3-yl)ethoxy)pyrimidin-4-yl)phenyl)propanoic acid;

(2S)-2-Amino-3-(4-(2-amino-6-(2,2,2-trifluoro-1-(1-phenyl-5-(trifluoromethyl)-1H-pyrazol-4-yl)ethoxy)pyrimidin-4-yl)phenyl)propanoic acid;

(2S)-2-Amino-3-(4-(2-amino-6-(1-(2-(cyclohexyloxy)-4-methylphenyl)-2,2,2-trifluoroethoxy)pyrimidin-4-yl)phenyl)propanoic acid;

(2S)-2-Amino-3-(4-(2-amino-6-(1-(2-(cyclopentyloxy)-4-methylphenyl)-2,2,2-trifluoroethoxy)pyrimidin-4-yl)phenyl)propanoic acid;

(2S)-2-Amino-3-(4-(2-amino-6-(1-(benzo[d]thiazol-6-yl)-2,2,2-trifluoroethoxy)pyrimidin-4-yl)phenyl)propanoic acid;

(2S)-2-Amino-3-(4-(2-amino-6-(2,2,2-trifluoro-1-(1-methyl-1H-imidazol-5-yl)ethoxy)pyrimidin-4-yl)phenyl)propanoic acid;

(2S)-2-Amino-3-(4-(6-(1-(2-(cyclopentyloxy)-4-methyl phenyl)-2,2,2-trifluoroethoxy)pyrimidin-4-yl)phenyl)propanoic acid;

(2S)-2-Amino-3-(4-(6-(1-(2-(cyclohexyloxy)-4-methylphenyl)-2,2,2-trifluoroethoxy)pyrimidin-4-yl)phenyl)propanoic acid;

(2S)-2-Amino-3-(4-(2-amino-6-(2,2,2-trifluoro-1-(pyridin-3-yl)ethoxy)pyrimidin-4-yl)phenyl)propanoic acid;

(2S)-2-Amino-3-(4-(2-amino-6-(1-(1,3-dimethyl-1H-pyrazol-5-yl)-2,2,2-trifluoroethoxy)pyrimidin-4-yl)phenyl)propanoic acid;

(S)-2-Amino-3-(4-(2-amino-6-(3-hydroxyphenyl)pyrimidin-4-yl)phenyl)propanoic acid;

(2S)-2-Amino-3-(4-(2-amino-6-(2,2,2-trifluoro-1-(3-hydroxybiphenyl-2-yl)ethoxy)pyrimidin-4-yl)phenyl)propanoic acid;

(S)-2-Amino-3-(4-(2-amino-6-(3,5-difluorophenyl)pyrimidin-4-yl)phenyl)propanoic acid;

(2S)-2-Amino-3-(4-(2-amino-6-(1-(3′,5′-difluorobiphenyl-2-yl)-2,2,2-trifluoroethoxy)pyrimidin-4-yl)phenyl)propanoic acid;

(2S)-2-Amino-3-(4-(6-(2,2,2-trifluoro-1-(3′-fluorobiphenyl-3-yl)ethoxy)pyrazin-2-yl)phenyl)propanoic acid;

(2S)-2-Amino-3-(4-(2-amino-6-(1-(5-ethoxy-2-methyl-2,3-dihydrobenzofuran-6-yl)-2,2,2-trifluoroethoxy)pyrimidin-4-yl)phenyl)propanoic acid;

(2S)-2-Amino-3-(4-(2-amino-6-(1-(benzofuran-5-yl)-2,2,2-trifluoroethoxy)pyrimidin-4-yl)phenyl)propanoic acid;

(2S)-2-Amino-3-(4-(2-amino-6-(2,2,2-trifluoro-1-(2-m-tolylfuran-3-yl)ethoxy)pyridin-4-yl)phenyl)propanoic acid;

(S)-Ethyl 3-(4-(2-amino-6-((R)-2,2,2-trifluoro-1-(3′-methoxybiphenyl-4-yl)ethoxy)pyrimidin-4-yl)phenyl)-2-(2-aminoacetamido)propanoate;

(2S)-2-Amino-3-(4-(6-(2,2,2-trifluoro-1-(2-(4-methylthiophen-3-yl)phenyl)ethoxy)pyrazin-2-yl)phenyl)propanoic acid;

(2S)-2-Amino-3-(4-(2-amino-6-(2,2,2-trifluoro-1-(5-methyl-3-phenylisoxazol-4-yl)ethoxy)pyrimidin-4-yl)phenyl)propanoic acid;

(S)-2-Amino-3-(4-(2-amino-6-(3-(methylthio)phenyl)pyrimidin-4-yl)phenyl)propanoic acid;

(2S)-2-Amino-3-(4-(2-amino-6-(2,2,2-trifluoro-1-(3′-(methylthio)biphenyl-2-yl)ethoxy)pyrimidin-4-yl)phenyl)propanoic acid;

(2S)-2-Amino-3-(4-(2-amino-6-(1-(3′-((dimethylamino)methyl)biphenyl-2-yl)-2,2,2-trifluoroethoxy)pyrimidin-4-yl)phenyl)propanoic acid;

(S)-2-Amino-3-(4-(2-amino-6-(3-(trifluoromethoxy)phenyl)pyrimidin-4-yl)phenyl)propanoic acid;

(2S)-2-Amino-3-(4-(2-amino-6-(2,2,2-trifluoro-1-(3′-(trifluoromethoxy)biphenyl-2-yl)ethoxy)pyrimidin-4-yl)phenyl)propanoic acid;

(S)-3-(4-(2-Amino-6-((R)-2,2,2-trifluoro-1-(3′-methoxybiphenyl-4-yl)ethoxy)pyrimidin-4-yl)phenyl)-2-(2-aminoacetamido)propanoic acid;

(2S)-2-Amino-3-(4-(2-amino-6-(2,2,2-trifluoro-1-(1-methyl-5-phenyl-1H-pyrazol-4-yl)ethoxy)pyrimidin-4-yl)phenyl)propanoic acid;

(2S)-2-Amino-3-(4-(2-amino-6-(2,2,2-trifluoro-1-(4-(methylsulfonyl)phenyl)ethoxy)pyrimidin-4-yl)phenyl)propanoic acid;

(S)-2-Amino-3-(4-(2-amino-6-((R)-1-(3′-(dimethylamino)biphenyl-2-yl)-2,2,2-trifluoroethoxy)pyrimidin-4-yl)phenyl)propanoic acid;

(2S)-2-Amino-3-(4-(2-amino-6-(1-(2-chloro-4-(methylsulfonyl)phenyl)-2,2,2-trifluoroethoxy)pyrimidin-4-yl)phenyl)propanoic acid;

(2S)-2-Amino-3-(4-(2-amino-6-(2,2,2-trifluoro-1-(3-(furan-2-yl)thiophen-2-yl)ethoxy)pyrimidin-4-yl)phenyl)propanoic acid;

(2S)-2-Amino-3-(4-(2-amino-6-(1-(2-(cyclopentyloxy)-4-fluorophenyl)-2,2,2-trifluoroethoxy)pyrimidin-4-yl)phenyl)propanoic acid;

(2S)-2-Amino-3-(4-(2-amino-6-(2,2,2-trifluoro-1-(2-(3-methoxyphenyl)cyclohex-1-enyl)ethoxy)pyrimidin-4-yl)phenyl)propanoic acid;

(2S)-2-Amino-3-(4-(2-amino-6-(2,2,2-trifluoro-1-(pyrimidin-5-yl)ethoxy)pyrimidin-4-yl)phenyl)propanoic acid;

(2S)-2-Amino-3-(4-(5-(2,2,2-trifluoro-1-(3′-methoxybiphenyl-3-yl)ethoxy)pyrazin-2-yl)phenyl)propanoic acid;

(S)-2-Amino-3-(4-(2-amino-6-((S)-1-(3′-(dimethylamino)biphenyl-2-yl)-2,2,2-trifluoroethoxy)pyrimidin-4-yl)phenyl)propanoic acid;

(2S)-2-Amino-3-(4-(2-amino-6-(2,2,2-trifluoro-1-(2-(furan-2-carboxamido)phenyl)ethoxy)pyrimidin-4-yl)phenyl)propanoic acid;

(2S)-2-Amino-3-(4-(2-amino-6-(1-(4-chloro-2-(methylsulfonyl)phenyl)-2,2,2-trifluoroethoxy)pyrimidin-4-yl)phenyl)propanoic acid;

(S)-Isopropyl 2-amino-3-(4-(2-amino-6-((R)-2,2,2-trifluoro-1-(3′-methoxybiphenyl-4-yl)ethoxy)pyrimidin-4-yl)phenyl)propanoate;

(2S)-2-Amino-3-(4-(6-(1-(2-(cyclopentyloxy)-4-fluorophenyl)-2,2,2-trifluoroethoxy)pyrimidin-4-yl)phenyl)propanoic acid;

(2S)-2-Amino-3-(4-(6-(1-(2-(cyclohexyloxy)-4-fluorophenyl)-2,2,2-trifluoroethoxy)pyrimidin-4-yl)phenyl)propanoic acid;

(2S)-2-Amino-3-(4-(2-amino-6-(2,2,2-trifluoro-1-(1-(thiophen-2-yl)cyclohexyl)ethoxy)pyrimidin-4-yl)phenyl)propanoic acid;

(2S)-2-Amino-3-(4-(2-(2,2,2-trifluoro-1-(3′-methoxybiphenyl-4-yl)ethoxy)thiazol-5-yl)phenyl)propanoic acid;

(2S)-2-Amino-3-(4-(2-amino-6-(1-(2-(cyclohexyloxy)-4-fluorophenyl)-2,2,2-trifluoroethoxy)pyrimidin-4-yl)phenyl)propanoic acid;

(2S)-2-Amino-3-(4-(2-amino-6-(2,2,2-trifluoro-1-(1-(4-methoxyphenyl)cyclohexyl)ethoxy)pyrimidin-4-yl)phenyl)propanoic acid;

(2S)-2-Amino-3-(4-(6-(2,2,2-trifluoro-1-(4-fluoro-2-methylphenyl)ethoxy)pyrimidin-4-yl)phenyl)propanoic acid;

(2S)-2-Amino-3-(4-(2-amino-6-(2,2,2-trifluoro-1-(4-fluoro-2-methylphenyl)ethoxy)pyrimidin-4-yl)phenyl)propanoic acid;

(2S)-2-Amino-3-(4-(2-amino-6-(oxazol-2-yl(phenyl)methoxy)pyrimidin-4-yl)phenyl)propanoic acid;

(S)-2-Amino-3-(4-(2-amino-6-(1-cyclohexyl-2,2,2-trifluoroethylideneaminooxy)pyrimidin-4-yl)phenyl)propanoic acid;

(2S)-2-Amino-3-(4-(2-amino-6-(1-(2-(3-(dimethylamino)phenyl)furan-3-yl)-2,2,2-trifluoroethoxy)pyrimidin-4-yl)phenyl)propanoic acid;

(2S)-2-Amino-3-(4-(2-amino-6-(2,2,2-trifluoro-1-(5-phenylthiophen-2-yl)ethoxy)pyrimidin-4-yl)phenyl)propanoic acid;

(S)-Phenyl 2-amino-3-(4-(2-amino-6-((R)-2,2,2-trifluoro-1-(3′-methoxybiphenyl-4-yl)ethoxy)pyrimidin-4-yl)phenyl)propanoate;

(S)-2-Amino-3-(4-(2-amino-6-((R)-1-(3′-((dimethylamino)methyl)biphenyl-4-yl)-2,2,2-trifluoroethoxy)pyrimidin-4-yl)phenyl)propanoic acid;

(S)-2-Amino-3-(4-(1-(3-methoxybenzoyl)-1H-pyrazol-4-yl)phenyl)propanoic acid;

(2S)-2-Amino-3-(4-(6-(2,2,2-trifluoro-1-(5-phenylfuran-2-yl)ethoxy)pyrimidin-4-yl)phenyl)propanoic acid;

(2S)-2-Amino-3-(4-(2-amino-6-(1-(4-chloro-2-fluorophenyl)-2,2,2-trifluoroethoxy)pyrimidin-4-yl)phenyl)propanoic acid;

(S,E)-2-Amino-3-(4-(2-amino-6-(4-(trifluoromethyl)styryl)pyrimidin-4-yl)phenyl)propanoic acid;

(2S)-2-Amino-3-(4-(2-amino-6-(1-(3,4-dichlorophenyl)-2,2,2-trifluoroethoxy)pyrimidin-4-yl)phenyl)propanoic acid;

(2S)-2-Amino-3-(4-(2-amino-6-(1-(4-chloro-3-fluorophenyl)-2,2,2-trifluoroethoxy)pyrimidin-4-yl)phenyl)propanoic acid;

(S)-2-Amino-3-(4-(2-amino-6-((R)-1-(3′-(dimethylamino)biphenyl-4-yl)-2,2,2-trifluoroethoxy)pyrimidin-4-yl)phenyl)propanoic acid;

(2S)-2-Amino-3-(4-(2-amino-6-(1-chloro-2,2,2-trifluoro-1-(4-methoxybiphenyl-2-yl)ethoxy)pyrimidin-4-yl)phenyl)propanoic acid;

(2S)-2-Amino-3-(4-(6-(2,2,2-trifluoro-1-(5-phenylthiophen-2-yl)ethoxy)pyrimidin-4-yl)phenyl)propanoic acid;

(S)-2-Amino-3-(4-(5-(4-phenoxyphenyl)-1H-1,2,3-triazol-1-yl)phenyl)propanoic acid;

(S,E)-2-Amino-3-(4-(2-amino-6-(2-(biphenyl-4-yl)vinyl)pyrimidin-4-yl)phenyl)propanoic acid;

(S)-2-Amino-3-(4-(4-amino-6-((R)-2,2,2-trifluoro-1-(3′-methoxybiphenyl-4-yl)ethoxy)pyrimidin-2-yl)phenyl)propanoic acid;

(S)-2-Amino-3-(4-(4′-methoxybiphenyl-4-ylsulfonamido)phenyl)propanoic acid;

(2S)-2-Amino-3-(4-(2-amino-6-(2,2,2-trifluoro-1-(6-(3-methoxyphenyl)pyridin-3- yl)ethoxy)pyrimidin-4-yl)phenyl)propanoic acid;

(2S)-2-Amino-3-(4-(2-amino-6-(2,2,2-trifluoro-1-(6-(2-fluoro-3-methoxyphenyl)pyridin-3-yl)ethoxy)pyrimidin-4-yl)phenyl)propanoic acid;

2-Amino-3-(5-(4′-methylbiphenyl-4-yl)-1H-indol-3-yl)propanoic acid;

2-Amino-3-(5-m-tolyl-1H-indol-3-yl)propanoic acid;

(2S)-2-Amino-3-(4-(2-(2-methoxyphenyl)furan-3-carboxamido)phenyl)propanoic acid;

2-Amino-3-(5-(1-benzyl-1H-pyrazol-4-yl)-1H-indol-3-yl)propanoic acid;

(2S)-2-Amino-3-(4-(2-amino-6-(2,2,2-trifluoro-1-(6-(thiophen-2-yl)pyridin-3-yl)ethoxy)pyrimidin-4-yl)phenyl)propanoic acid;

2-Amino-3-(6-(1-benzyl-1H-pyrazol-4-yl)-1H-indol-3-yl)propanoic acid;

(S)-2-Amino-3-(4-((2-(4-(trifluoromethyl)phenyl)thiazol-4-yl)methylamino)phenyl)propanoic acid;

(S)-2-Amino-3-(4-((4′-methoxybiphenyl-4-ylsulfonamido)methyl)phenyl)propanoic acid;

(S)-2-Amino-3-(4-(3-(2-methoxydibenzo[b,d]furan-3-yl)-ureido)phenyl)propanoic acid;

(S)-2-Amino-3-(4-(3-(2,2-diphenylethyl)ureido)phenyl)propanoic acid;

(S)-2-Amino-3-(4-(phenylethynyl)phenyl)propanoic acid;

(S)-2-Amino-3-(4-(2-amino-6-((5-(1-methyl-5-(trifluoromethyl)-1H-pyrazol-3-yl)thiophen-2-yl)methoxy)pyrimidin-4-yl)phenyl)propanoic acid;

(2S)-2-Amino-3-(4-(2-amino-6-(1,1,1-trifluoro-3-((R)-2,2,3-trimethylcyclopent-3-enyl)propan-2-yloxy)pyrimidin-4-yl)phenyl)propanoic acid;

(2S)-2-Amino-3-(4-(2-amino-6-(3-(2-hydroxyethylcarbamoyl)piperidin-1-yl)pyrimidin-4-yl)phenyl)propanoic acid;

(2S)-2-Amino-3-(4-(2-amino-6-(3-(pyridin-2-yloxy)piperidin-1-yl)pyrimidin-4-yl)phenyl)propanoic acid;

(S)-2-Amino-3-(4-{2-amino-6-(4-chloro-3-(piperidine-1-carbonyl)phenyl)pyrimidin-4-yl}phenyl)propanoic acid;

(S)-2-Amino-3-(4-{2-amino-6-[2,2,2-trifluro-1-(4-pyridin-4-yl-phenyl)-ethoxy]-pyrimidin-4-yl}-phenyl)-propionic acid;

(S)-2-Amino-3-(4-{6-[2,2,2-trifluro-1-(2-pyridin-4-yl-phenyl)-ethoxy]-pyrimidin-4-yl}-phenyl)-propionic acid;

(S)-2-Amino-3-(4-{2-amino-6-[2,2,2-trifluro-1-(2-(4-methyl-thiophen-3-yl)-phenyl]ethoxy}-pyrimidin-4-yl)-phenyl]-propionic acid;

(S)-2-Amino-3-(4-{2-amino-6-[2,2,2-trifluro-1-(2-(5-methyl-thiophen-3-yl)-phenyl]ethoxy}-pyrimidin-4-yl)-phenyl]-propionic acid;

(S)-2-Amino-3-(4-{2-amino-6-[2,2,2-trifluoro-1-(4-furan-3-yl-phenyl)-ethoxy]-pyrimidin-4-yl}-phenyl)-propionic acid;

(S)-2-Amino-3-[4-{2-amino-6-{1-2-(5-dimethylaminomethyl-furan-2-yl)-phenyl]-2,2,2-trifluoro-ethoxy}-pyrimidin-4-yl)-phenyl]-propionic acid;

(S)-2-Amino-3[4-(2-amino-6-{1-[2-(6-cyano-pyridin-3-yl)-phenyl]-2,2,2-trifluoro-ethoxy}-pyrimidin-4-yl)-phenyl]propionic acid;

(S)-2-Amino-3-(4-[2-amino-6-[2,2,2-trifluoro-1-(2-imidazol-1-yl-phenyl)-ethoxy]-pyrimidin-4-yl}-phenyl)-propionic acid;

(S)-2-Amino-3-(4-{6-[2,2,2-trifluoro-1-(2-pyrazol-1-yl-phenyl)-ethoxy]-pyrimidin-4-yl}-phenyl)-propionic acid;

(S)-2-amino-3-[4-(2-amino-6-{2,2,2-trifluoro-1-[2-(3-trifluoromethyl-pyrazol-1-yl)-phenyl]-ethoxy}-pyrimidin-4-yl)-phenyl[-propionic acid;

(S)-2-Amino-3-[4-(2-amino-6-{1-[2-(3,5-dimethyl-pyrazol-1-yl)-phenyl]-2,2,2-trifluoro-ethoxy}-pyrimidin-4-yl)phenyl]propionic acid;

(S)-2-Amino-3-[4-(2-amino-6-{2,2,2-trifluoro-1-[2-(3-phenyl-pyrazol-1-yl)-phenyl]-ethoxy}-pyrimidin-4-yl)-phenyl]propionic acid;

(S)-2-Amino-3-[4-(2-amino-6-{2,2,2-trifluoro-1-[5-methoxy-2-(4-methyl-pyrazol-1-yl)-phenyl]-ethoxy}-pyrimidin-4-yl)-phenyl]propionic acid;

(S)-2-amino-3-[4-(2-amino-6-{(R)-2,2,2-trifluoro-1-[2-(3-methyl-pyrazol-1-yl)-phenyl]-ethoxy}-pyrimidin-4-yl)-phenyl]propionic acid;

(S)-2-amino-3-[4-(2-amino-6-{1-[4-chloro-2-(3-methyl-pyrazol-1-yl)-phenyl]-2,2,2-trifluoro-ethoxy}-pyrimidin-4-yl)-phenyl]propionic acid;

(S)-2-Amino-3-[4-(2-amino-6-{R-1-[4-chloro-2-(3-methyl-pyrazol-1-yl)-phenyl]-2,2,2-trifluoro-ethoxy}-pyrimidin-4-yl)-phenyl]propionic acid ethyl ester;

(S)-2-amino-3-(4-(2-amino-6-{(R)-1-(4-chloro-2-(3-methyl-1H-pyrazol-1-yl)phenyl)-2,2,2- trifluoroethoxy)pyrimidin-4-yl)phenyl)propanoic acid;

(S)-2-Amino-3-(4-{2-amino-6-[2,2,2-trifluoro-1-(2-thiazol-2-yl-phenyl)-ethoxy]-pyrimidin-4-yl}- phenyl)-propionic acid;

(S)-2-Amino-3-[4-(2-amino-6-{2,2,2-trifluoro-1-[2-(pyridin-3-yloxy)-phenyl]-ethoxy}-pyrimidin-4-yl)phenyl]propionic acid;

(S)-2-Amino-3-[4-(2-amino-6-{2,2,2-trifluoro-1-[4-(pyridin-3-yloxy)-phenyl]-ethoxy}-pyrimidin-4-yl)-phenyl]propionic acid;

(S)-2-Amino-3-[4-(6-{2,2,2-trifluoro-1-[4-(pyridin-3-yloxy)-phenyl]-ethoxy}-pyrimidin-4-yl)-phenyl]-propionic acid;

(S)-2-Amino-3-[4-{2-amino-6-[2,2,2-trifluoro-1-(4-thiophen-2-yl-phenyl)-ethoxy]-pyrimidin-4-yl}-phenyl)-propionic acid;

(S)-2-Amino-3-(4-{6-[2,2,2-trifluoro-1-(4-imidazol-1-yl-phenyl)-ethoxy]-pyrimidin-4-yl}-phenyl)-propionic acid;

(S)-2-Amino-3-(4-{2-amino-6-[2,2,2-trifluoro-1-(4-[1,2,4]triazol-1-yl-phenyl)-ethoxy]-pyrimidin-4-yl}-phenylypropionic acid;

(S)-2-Amino-3-(4-{2-amino-6-[2,2,2-trifluoro-1-(4-fluoro-2-thiophen-3-yl-phenyl)ethoxy]pyrimidin-4-yl}-phenyl)-propionic acid;

(S)-2-Amino-3-[4-(2-amino-6-{2,2,2-trifluoro-1-[4-fluoro-2-(4-methyl-thiophen-2-yl)-phenyl]-ethoxy}-pyrimidin-4-yl)-phenyl]propionic acid;

(S)-2-Amino-3-[4-(2-amino-6-{1-[2-(3,5-dimethyl-isoxazol-4-yl)-4-fluoro-phenyl]-2,2,2-trifluoro-ethoxy}-pyrimidin-4-yl)-phenyl]propionic acid;

(S)-2-amino-3-[4-(2-amino-6-{2,2,2-trifluoro-1-[5-fluoro-2-(3-methyl-pyrazol-1-yl)-phenyl[-ethoxy}-pyrimidin-4-yl)-phenyl]propionic acid;

(S)-2-amino-3-[4-(2-amino-6{2,2,2-trifluoro-1-[5-chloro-2-(3-methyl-pyrazol-1-yl)-phenyl]-ethoxy}-pyrimidin-4-yl)-phenyl]-propionic acid;

(S)-2-amino-3-[4-(2-amino-6-{2,2,2-trifluoro-1-[4-(2-oxo-pyrrolidin-1-yl)-phenyl]-ethoxy}-pyrimidin-4-yl)-phenyl]propionic acid;

(S)-2-Amino-3-[4-(2-amino-6-{(R)-2,2,2-trifluoro-1-[5-fluoro-2-(3-methyl-pyrazol-1-yl)-phenyl]-ethoxy}-pyrimidin-4-yl)-phenyl]propionic acid;
(S)-2-Amino-3-[4-(2-amino-6-[2,2,2-trifluoro-1-[4-(6-methoxy-pyridin-2-yl)-phenyl]-ethoxy}-pyrimidin-4-yl)-phenyl]propionic acid;

(S)-2-Amino-3-[4-(2-amino-6-{2,2,2-trifluoro-1-[2-fluoro-4-(5-methoxy-pyridin-3-yl)-phenyl]-ethoxy}-pyrimidin-4-yl)-phenyl]propionic acid;

(S)-2-Amino-3-[4-(2-amino-6-{(S)-2,2,2-trifluoro-1-[4-(2-fluoro-pyridin-4-yl)-phenyl]-ethoxy}-pyrimidin-4-yl)-phenyl]-propionic acid;

(S)-2-Amino-3-[4-(2-amino-6-{(S)-2,2,2-trifluoro-1-[4-(5-methoxy-pyridin-3yl)-phenyl]- ethoxy}-pyrimidin-4-yl)-phenyl]propionic acid;

(S)-2-Amino-3-[4-(2-amino-6-[(S)-2,2,2-trifluoro-1-[4-(4-trifluoromethyl-pyridin-3-yl)-phenyl]-ethoxy}-pyrimidin-4-yl)-phenyl]propionic acid; (S)-2-Amino-3-(442-amino-6-[(S)-2,2,2-trifluoro-1-(4-isoxazol-4-yl-phenyl)-ethoxy]-pyrimidin-4-yl}-phenyl)-propionic acid;

(S)-2-Amino-3-(4-{2-amino-6-[2,2,2-trifluoro-1-(2-pyrimidin-5-yl-phenyl)-ethoxy]-pyrimidin-4-yl}-phenyl)-propionic acid;

(S)-2-amino-3-(4-[2-amino-6-[2,2,2-trifluoro-1-(2-thiophen-3-yl-phenyl)-ethoxy]-pyrimidin-4-yl}-phenyl)-propionic acid;

(S)-2-Amino-3-[4-(2-amino-6-[2,2,2-trifluoro-1-[2-(1-methyl-1H-pyrazol-4-yl)-phenyl]- ethoxy}-pyrimidin-4-yl)-phenyl]propionic acid;

(S)-2-amino-3-(4-{6-[2,2,2-trifluoro-1-(2-furan-3-yl-phenyl)-ethoxy]-pyrimidin-4-yl}-phenyl)-propionic acid;

(S)-2-amino-3-(4-{6-[2,2,2-trifluoro-1-(2-furan-2-yl-phenyl)-ethoxy]-pyrimidin-4-yl}-phenyl)-propionic acid;

(2S)-2-amino-3-(4-(2-amino-6-(2,2,2-trifluoro-1-(4-(pyridin-3-yl)phenyl)ethoxy)pyrimidin-4-yl)phenyl)propanoic acid;

(2S)-2-amino-3-(4-(2-amino-6-(2,2,2-trifluoro-1-(2-(2-methylpyridin-4-yl)phenyl)ethoxy)pyrimidin-4-yl)phenyl)propanoic acid;

(2S)-2-amino-3-(4-(2-amino-6-(2,2,2-trifluoro-1-(2-(4-methylthiophen-3-yl)phenyl)ethoxy)pyrimidin-4-yl)phenyl)propanoic acid;

(2S)-3-(4-(6-(1-(2-(1H-pyrazol-1-yl)phenyl)-2,2,2-trifluoroethoxy)-2-aminopyrimidin-4-yl)phenyl)-2-aminopropanoic acid;

(2S)-2-amino-3-(4-(2-amino-6-(2,2,2-trifluoro-1-(4-(furan-2-yl)phenyl)ethoxy)pyrimidin-4-yl)phenyl)propanoic acid;

(2S)-2-amino-3-(4-(6-(2,2,2-trifluoro-1-(2-(pyridin-3-yloxy)phenyl)ethoxy)pyrimidin-4-yl)phenyl)propanoic acid;

(2S)-3-(4-(6-(1-(2-(1H-1,2,4-triazol-1-yl)phenyl)-2,2,2-trifluoroethoxy)-2-aminopyrimidin-4-yl)phenyl)-2-aminopropanoic acid;

(2S)-2-amino-3-(4-(2-amino-6-(2,2,2-trifluoro-1-(2-(furan-3-yl)phenyl)ethoxy)pyrimidin-4-yl)phenyl)propanoic acid;

(2S)-2-amino-3-(4-(2-amino-6-(2,2,2-trifluoro-1-(4-(furan-2-yl)-3-methoxyphenyl)ethoxy)pyrimidin-4-yl)phenyl)propanoic acid;

(2S)-2-amino-3-(4-(5-(2,2,2-trifluoro-1-(2-(furan-2-yl)phenyl)ethoxy)pyrazin-2-yl)phenyl)propanoic acid;

(2S)-3-(4-(5-(1-(2-(1H-pyrazol-1-yl)phenyl)-2,2,2-trifluoroethoxy)pyrazin-2-yl)phenyl)-2-aminopropanoic acid;

(2S)-2-amino-3-(4-(2-amino-6-(1-(4,5-dimethoxy-2-(1H-pyrazol-1-yl)phenyl)-2,2,2-trifluoroethoxy)pyrimidin-4-yl)phenyl)propanoic acid;

(2S)-2-amino-3-(4-(2-amino-6-(2,2,2-trifluoro-1-(2-(2-methyl-1H-imidazol-1-yl)phenyl)ethoxy)pyrimidin-4-yl)phenyl)propanoic acid;

(2S)-2-amino-3-(4-(2-amino-6-(2,2,2-trifluoro-1-(2-(5-methylthiophen-2-yl)phenyl)ethoxy)pyrimidin-4-yl)phenyl)propanoic acid;

(2S)-2-amino-3-(4-(2-amino-6-(1-(2-(5-(dimethylcarbamoyl)furan-2-yl)phenyl)-2,2,2-trifluoroethoxy)pyrimidin-4-yl)phenyl)propanoic acid;

(2S)-2-amino-3-(4-(2-amino-6-(2,2,2-trifluoro-1-(4-fluoro-2-(thiophen-2-yl)phenyl)ethoxy)pyrimidin-4-yl)phenyl)propanoic acid;

(2S)-2-amino-3-(4-(6-(2,2,2-trifluoro-1-(4-fluoro-2-(thiophen-2-yl)phenyl)ethoxy)pyrimidin-4-yl)phenyl)propanoic acid;

(2S)-2-amino-3-(4-(6-(2,2,2-trifluoro-1-(4-fluoro-2-(thiophen-3-yl)phenyl)ethoxy)pyrimidin-4-yl)phenyl)propanoic acid;

(2S)-2-amino-3-(4-(6-(2,2,2-trifluoro-1-(4-fluoro-2-(4-methylthiophen-2-yl)phenyl)ethoxy)pyrimidin-4-yl)phenyl)propanoic acid;

(S)-2-amino-3-(4-(2-amino-6-((R)-2,2,2-trifluoro-1-(4-(6-fluoropyridin-3-yl)phenyl)ethoxy)pyrimidin-4-yl)phenyl)propanoic acid;

(2S)-3-(4-(6-(1-(4-(1H-imidazol-1-yl)phenyl)-2,2,2-trifluoroethoxy)-2-aminopyrimidin-4-yl)phenyl)-2-aminopropanoic acid;

(2S)-2-amino-3-(4-(6-(2,2,2-trifluoro-1-(4-(thiophen-2-yl)phenyl)ethoxy)pyrimidin-4-yl)phenyl)propanoic acid;

(S)-2-amino-3-(4-(2-amino-6-((R)-2,2,2-trifluoro-1-(4-(pyrimidin-5-yl)phenyl)ethoxy)pyrimidin-4-yl)phenyl)propanoic acid;

(2S)-2-amino-3-(4-(6-(1-(2-(3,5-dimethylisoxazol-4-yl)-4-fluorophenyl)-2,2,2-trifluoroethoxy)pyrimidin-4-yl)phenyl)propanoic acid;

(S)-2-amino-3-(4-(2-amino-6-((R)-2,2,2-trifluoro-1-(4-(2-methylpyridin-4-yl)phenyl)ethoxy)pyrimidin-4-yl)phenyl)propanoic acid;

(2S)-3-(4-(6-(1-(4-(1H-1,2,4-triazol-1-yl)phenyl)-2,2,2-trifluoroethoxy)pyrimidin-4-yl)phenyl)-2-aminopropanoic acid;

(2S)-2-amino-3-(4-(2-amino-6-(2,2,2-trifluoro-1-(4-(piperidin-1-ylmethyl)phenyl)ethoxy)pyrimidin-4-yl)phenyl)propanoic acid;

(2S)-2-amino-3-(4-(2-amino-6-(2,2,2-trifluoro-1-(2-fluoro-4-(2-methylpyridin-4-yl)phenyl)ethoxy)pyrimidin-4-yl)phenyl)propanoic acid;

(2S)-2-amino-3-(4-(2-amino-6-(1-(4-(6-chloropyridazin-3-yl)phenyl)-2,2,2-trifluoroethoxy)pyrimidin-4-yl)phenyl)propanoic acid;

(2S)-2-amino-3-(4-(2-amino-6-(1-(4-(4-tert-butylthiazol-2-yl)phenyl)-2,2,2-trifluoroethoxy)pyrimidin-4-yl)phenyl)propanoic acid;

(2S)-2-amino-3-(4-(2-amino-6-(2,2,2-trifluoro-1-(3′-methoxy-3-(3-methyl-1H-pyrazol-1-yl)biphenyl-4-yl)ethoxy)pyrimidin-4-yl)phenyl)propanoic acid;

(S)-ethyl-2-amino-3-(4-(2-amino-6-((R)-1-(4-chloro-2-(3-methyl-1H-pyrazol-1yl)phenyl)-2,2,2-trifluoroethoxy)pyrimidin-4-yl)phenyl)propanoate;

(2S)-2-amino-3-(4-(2-amino-6-(1-(5-chloro-2-(3-methyl-1H-pyrazol-1-yl)phenyl)-2,2,2-trifluoroethoxy)pyrimidin-4-yl)phenyl)propanoic acid;

and pharmaceutically acceptable salts thereof.

TPH inhibitors are preferably administered in pharmaceutical compositions suitable for administration to patients. Pharmaceutical compositions include those suitable for oral, mucosal (e.g., nasal, sublingual, vaginal, buccal, or rectal), parenteral (e.g., subcutaneous, intravenous, bolus injection, intramuscular, or intraarterial), transdermal, topical and ophthalmic (e.g., topical, intravitreal) administration.

Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

### 5.3. Assays, Kits and Methods of Treatment

This invention encompasses methods of treating IBS patients who are more likely to respond to TPH inhibitor therapy than not. Applicants have discovered that for non-constipating IBS patient, the higher their baseline 5-HT or 5-HIAA levels, the more likely they are to respond to TPH inhibitory therapy. Baseline levels are levels determined prior to therapy. Response can be measured by global relief of symptoms or by stool consistency.

One embodiment of the invention encompasses a method of treating or managing irritable bowel syndrome (IBS), which comprises administering to a patient suffering from IBS a therapeutically or prophylactically effective amount of a TPH inhibitor, wherein the patient suffering from IBS exhibits a baseline peripheral 5-HT level that is greater than the average peripheral 5-HT level exhibited by people who do not suffer from IBS. In particular methods, the peripheral 5-HT level is determined by measuring 5-HT in whole blood or plasma. In some, the measurement of 5-HT is made before a meal. In others, the measurement of 5-HT is made about 0.5, 1.0, 2.0, 3.0, or 4.0 hours after a meal. In some, the measurement of 5-HT is an average of a plurality of measurements taken over a period of time (e.g., 24, 48, 72 hours). In some, the baseline peripheral 5-HT level of the patient is at least about 10, 15, 20, 25, 30, 35, or 40 percent greater than the average peripheral 5-HT level exhibited by people who do not suffer from IBS.

Another embodiment encompasses a method of treating or managing IBS, which comprises administering to a patient suffering from IBS a therapeutically or prophylactically effective amount of a TPH inhibitor, wherein the patient suffering from IBS exhibits a baseline peripheral 5-HIAA level that is greater than the average peripheral 5-HIAA level exhibited by people who do not suffer from IBS. In some methods, the 5-HIAA level is determined by measuring urinary 5-HIAA. In some, the measurement of 5-HIAA is obtained from urine collected over at least about 12, 24, or 48 hours. In some, the baseline peripheral 5-HIAA level of the patient is at least about 10, 15, 20, 25, 30, 35, or 40 percent greater than the average peripheral 5-HIAA level exhibited by people who do not suffer from IBS.

In some methods, the peripheral 5-HIAA level is determined by measuring 5-HIAA in plasma. In some, the measurement of 5-HIAA is made before a meal. In others, the measurement of 5-HIAA is made about 0.5, 1.0, 2.0, 3.0, or 4.0 hours after a meal. In some, the measurement of 5-HIAA is an average of a plurality of measurements taken over a period of time (e.g., 24, 48, 72 hours).

Another embodiment encompasses a method of treating or IBS, which comprises administering to a patient suffering from IBS a therapeutically or prophylactically effective amount of a TPH inhibitor, wherein the patient exhibits a blood 5-HT level that is greater than about 140, 145, 150, 155, or 160 ng/mL. In some methods, the blood 5-HT level is measured before a meal. In others, the blood 5-HT level is measured about 0.5, 1.0, 2.0, 3.0, or 4.0 hours after a meal.

Another embodiment encompasses a method of treating or managing IBS, which comprises administering to a patient suffering from irritable bowel syndrome a therapeutically or prophylactically effective amount of a TPH inhibitor, wherein the patient exhibits a baseline urinary 5-HIAA level of greater than about 3.5. 3.75, 4.0, 4.25, 4.5 or 4.75 mg/day.

Another embodiment encompasses a method of treating or managing IBS, which comprises administering to a patient suffering from IBS an amount of a TPH inhibitor sufficient to lower the patient's blood 5-HT level by greater than about 10, 20, 30 or 40 ng/mL compared to baseline (e.g., the level measured within 1, 2 or 3 weeks prior to the start of treatment). In some methods, the peripheral 5-HT level is measured before a meal. In others, the peripheral 5-HT level is measured about 0.5, 1.0, 2.0, 3.0, or 4.0 hours after a meal.

Another embodiment encompasses a method of treating or managing IBS, which comprises administering to a patient suffering from IBS an amount of a TPH inhibitor sufficient to lower the patient peripheral 5-HIM level by greater than about 10, 20, 30 or 40 percent compared to baseline (e.g., the level measured within 1, 2 or 3 weeks prior to the start of treatment). In some methods, the peripheral 5-HIAA level is urinary 5-HIAA (e.g., determined using urine collected over at least 12, 24, or 48 hours). In others, the peripheral 5-HIAA is plasma 5-HIAA, and the amount of TPH inhibitor is sufficient to lower the patient's plasma 5-HIM level by at least about 2, 2.5, or 3 ng/mL compared to baseline, and/or by at least 10, 20, or 30 percent compared to baseline.

In some methods of the invention, the TPH inhibitor is administered orally. In some, the TPH inhibitor is administered twice (BID) or three times (TID) daily.

This invention also encompasses methods and kits for determining whether an IBS patient is likely to respond to TPH inhibitor therapy. These utilize methods of assaying for 5-HT and 5-HIAA, which are well known. See, e.g., Atkinson, W. et al., Gastroenterology 130:34-43, 34 (2006); Liu, Q. et al., JPET 325:47-55 (2008).

One embodiment of the invention encompasses a method of determining if a patient suffering from IBS will be responsive to TPH inhibitor therapy, which comprises determining: a) if the patient experiences abdominal pain or discomfort at least 3 days/month, which is associated with two or more of: improvement with defecation, onset associated with a change in frequency of stool, or onset associated with a change in form (appearance) of stool; and b) if the patient's plasma 5-HT level is greater than about 140, 145, 150, 155, or 160 ng/mL.

Another embodiment encompasses a method of determining if a patient suffering from IBS will be responsive to TPH inhibitor therapy, which comprises determining: a) if the patient experiences abdominal pain or discomfort at least 3 days/month, which is associated with two or more of: improvement with defecation, onset associated with a change in frequency of stool, or onset associated with a change in form (appearance) of stool; and b) if the patient's urinary 5-HIAA level is greater than about 3.5. 3.75, 4.0, 4.25, 4.5 or 4.75 mg/day.

Another embodiment encompasses a method of estimating the likelihood that a patient suffering from IBS will be responsive to TPH inhibitor therapy, which comprises: measuring the amount of 5-HT in the plasma of the patient; and correlating that amount with a TPH inhibitor responder rate, wherein the TPH inhibitor responder rate is a predicted likelihood of relief from IBS symptoms for patients having a baseline 5-HT level that falls within a predetermined range. In some methods, the TPH inhibitor responder rate for the predetermined range is the percent of IBS patients (e.g., patients in a blinded clinical study of the TPH inhibitor) who previously reported experiencing relief from IBS symptoms and who had baseline 5-HT levels that fell within the predetermined range. In some methods, the TPH inhibitor responder rate for the predetermined range is the percent of IBS patients (e.g., patients in a blinded clinical study of the TPH inhibitor) who previously reported a decrease in mean stool consistency (e.g., as measured on the Bristol Stool Scale) and who had baseline 5-HT levels that fell within the predetermined range.

Another embodiment encompasses a method of estimating the likelihood that a patient suffering from IBS will be responsive to TPH inhibitor therapy, which comprises: measuring the amount of 5-HIAA in the urine or plasma of the patient; and correlating that amount with a TPH inhibitor responder rate, wherein the TPH inhibitor responder rate is a predicted likelihood of relief from IBS symptoms for patients having a baseline 5-HIAA level that falls within a predetermined range. In some methods, the TPH inhibitor responder rate for the predetermined range is the percent of IBS patients (e.g., patients in a blinded clinical study of the TPH inhibitor) who previously reported experiencing relief from IBS symptoms and who had baseline 5-HIM levels that fell within the predetermined range. In some methods, the TPH inhibitor responder rate for the predetermined range is the percent of IBS patients (e.g., patients in a blinded clinical study of the TPH inhibitor) who previously reported a decrease in mean stool consistency (e.g., as measured on the Bristol Stool Scale) and who had baseline 5-HIAA levels that fell within the predetermined range.

In some methods of the invention, 5-HT levels are measured by ELISA. In others, they are measured by chromatography, e.g., liquid chromatography.

In some methods of the invention, 5-HIAA levels are measured by ELISA. In others, they are measured by chromatography, e.g., liquid chromatography. One embodiment of the invention encompasses a kit for determining whether a patient suffering from IBS will be responsive to TPH inhibitor therapy, which comprises: a device for measuring plasma 5-HT concentrations; and information that correlates or provides instructions as to how to correlate measurements obtained using the device with a TPH inhibitor responder rate, wherein the TPH inhibitor responder rate is a predicted likelihood of relief from IBS symptoms for patients having a baseline 5-HT level that falls within a predetermined range.

Some kits further comprises a questionnaire that can be used to determine whether the patient has IBS-D or IBS-A (e.g., whether the patient satisfies Rome III criteria). In some kits, the device utilizes an ELIZA. In some kits, the TPH inhibitor responder rate for the predetermined range of 5-HT levels is the percent of IBS patients (e.g., patients in a blinded clinical study of the TPH inhibitor) who previously reported experiencing relief from IBS symptoms and who had baseline 5-HT levels that fell within that predetermined range. In some kits, the TPH inhibitor responder rate for the predetermined range of 5-HT levels is the percent of IBS patients (e.g., patients in a blinded clinical study of the TPH inhibitor) who previously reported a decrease in mean stool consistency (e.g., as measured on the Bristol Stool Scale) and who had baseline 5-HT levels that fell within that predetermined range.

Another embodiment encompasses a kit for determining whether a patient suffering from IBS will be responsive to TPH inhibitor therapy, which comprises: a device for measuring urinary or blood (e.g., plasma) 5-HIAA concentrations; and information that correlates or provides instructions as to how to correlate measurements obtained using the device with a TPH inhibitor responder rate, wherein the TPH inhibitor responder rate is a predicted likelihood of relief from IBS symptoms for patients having a baseline 5-HIM level that falls within a predetermined range. Some kits further comprises a questionnaire that can be used to determine whether the patient has IBS-D or IBS-A (e.g., whether the patient satisfies Rome III criteria). In some kits, the device utilizes an ELIZA. In some, the TPH inhibitor responder rate for the predetermined range of 5-HIAA levels is the percent of IBS patients (e.g., patients in a blinded clinical study of the TPH inhibitor) who previously reported experiencing relief from IBS symptoms and who had baseline 5-HIAA levels that fell within that predetermined range. In some, the TPH inhibitor responder rate for the predetermined range of 5-HIAA levels is the percent of IBS patients (e.g., patients in a blinded clinical study of the TPH inhibitor) who previously reported a decrease in mean stool consistency (e.g., as measured on the Bristol Stool Scale) and who had baseline 5-HIAA levels that fell within that predetermined range.

### 6. EXAMPLES

Aspects of this invention can be understood from the following examples, which do not limit its scope.

### 6.1. Treatment of IBS Patients Using (S)-2-amino-3-(4-(2-amino-6-((R)-2,2,2-trifluoro-1-(3′-methoxybiphenyl-4-yl)ethoxy)pyrimidin-4-yl)phenyl)propanoic acid

A randomized, double-blind, placebo-controlled phase 2a clinical trial was conducted to assess the safety and efficacy of a TPH inhibitor, (S)-2-amino-3-(4-(2-amino-6-((R)-2,2,2-trifluoro-1-(3′-methoxybiphenyl-4-yl)ethoxy)pyrimidin-4-yl)phenyl)propanoic acid, in 155 patients suffering from IBS-D or IBS-A, based on Rome III criteria. Capsules containing the compound or placebo were orally administered. Two dose levels were tested: 250 mg QID and 1000 mg QID. A two-week run-in period was used to establish baseline symptoms, followed by a 28 day randomized, double-blind treatment period. There was then a two-week follow-up period.

During the study, patients' urine and blood were obtained to assess 5-HIAA and 5-HT levels. Methods used to assess clinical endpoints were: 1) global assessment by weekly questioning regarding adequate relief of IBS pain and discomfort; and 2) Bristol Stool Scale was used to assess stool consistency by daily automated telephonic IVRS contact.

It was found that patients randomized to the high dose arm showed a statistically significant improvement versus placebo in the weekly global assessment. The patients also showed significant improvement in stool consistency. These observations correlated with a reduction in blood 5-HT levels as well as 24 hour urinary 5-HIAA levels. Even more significant was an observed correlation between the baseline serotonin levels (i.e., as determined by measuring 5-HIAA levels in urine collected over a 24 hour period prior to dosing) of patients and their improvement in global response. In particular, the drug exerted a greater beneficial effect in IBS patients who had comparatively high baseline levels than in those who did not.

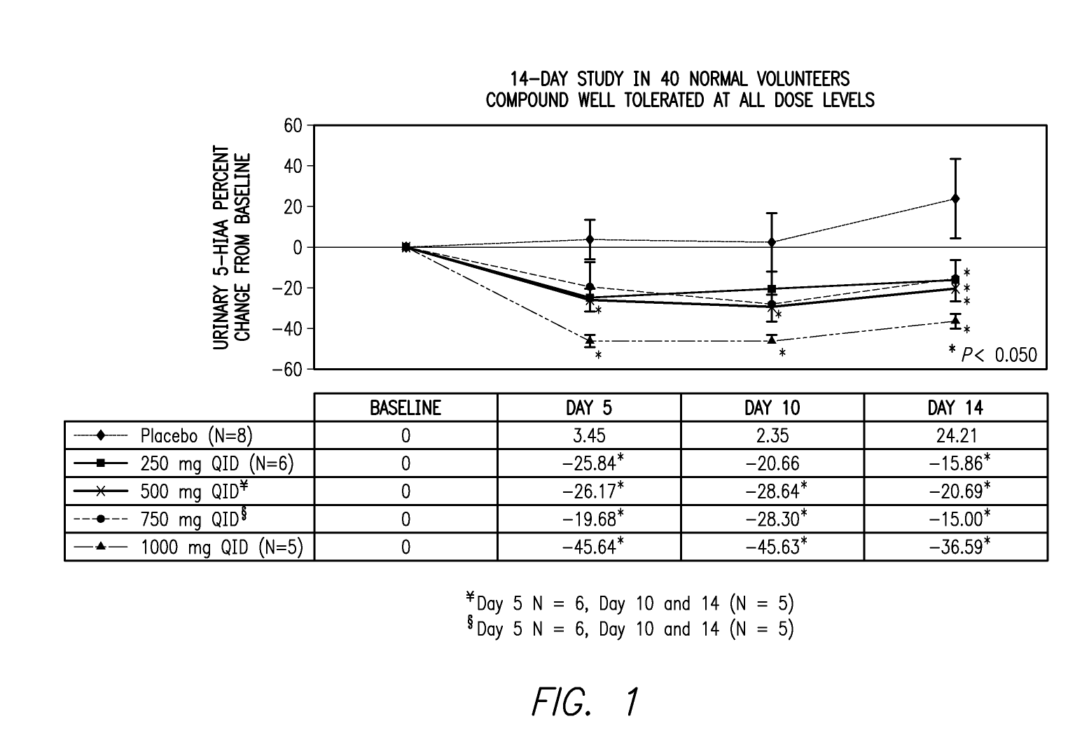

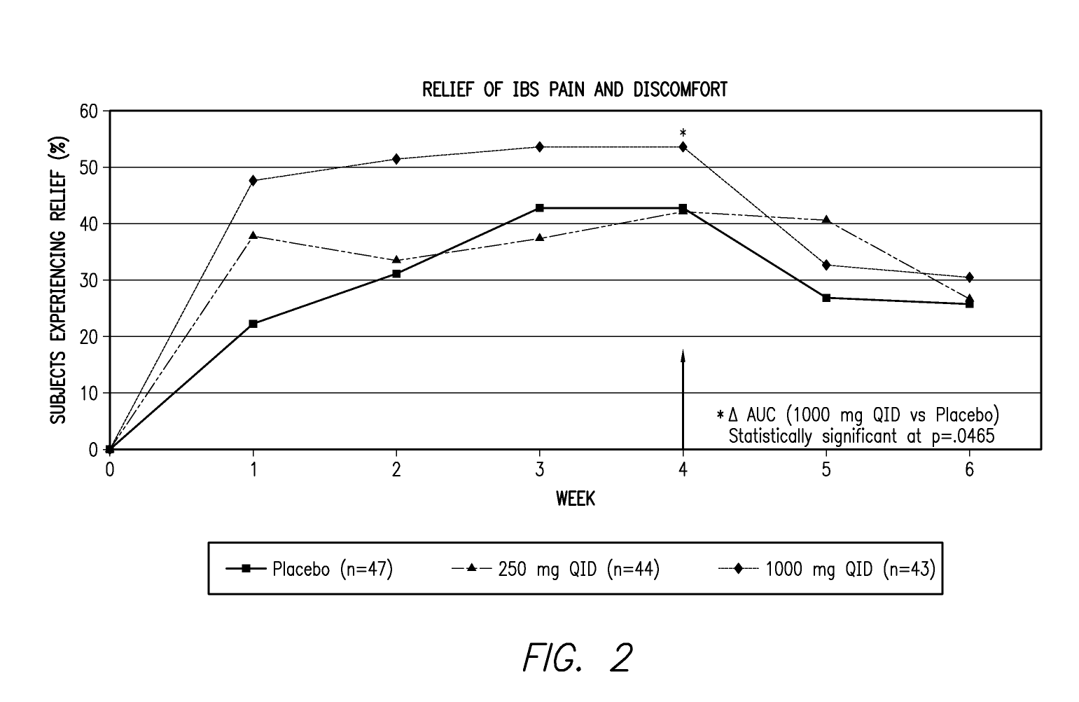

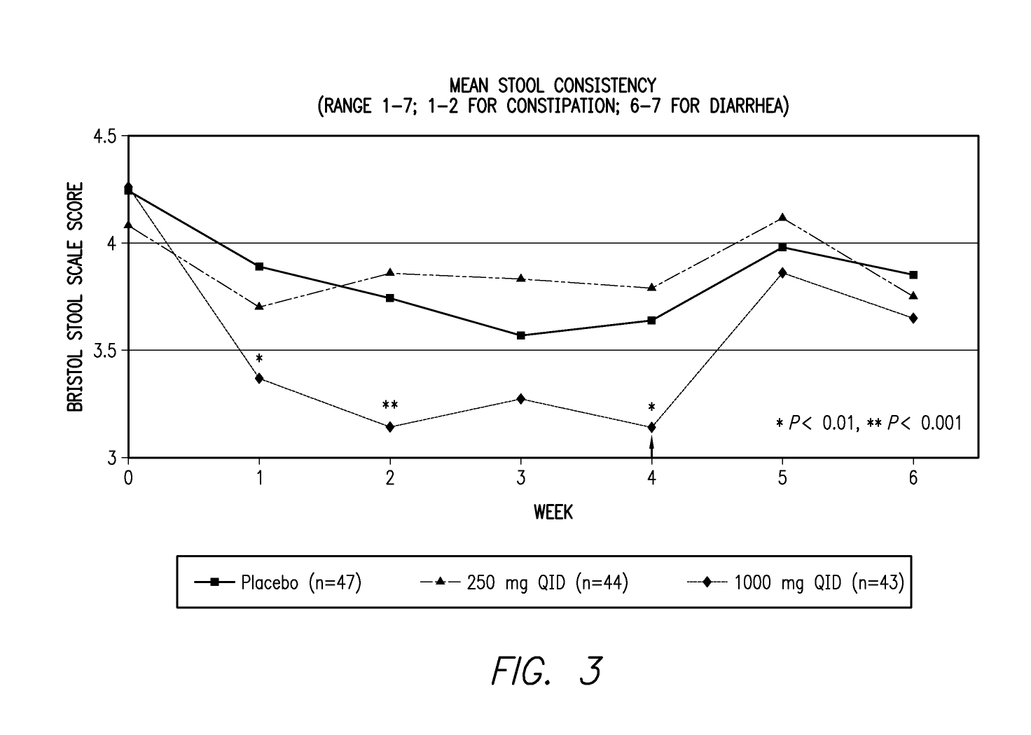

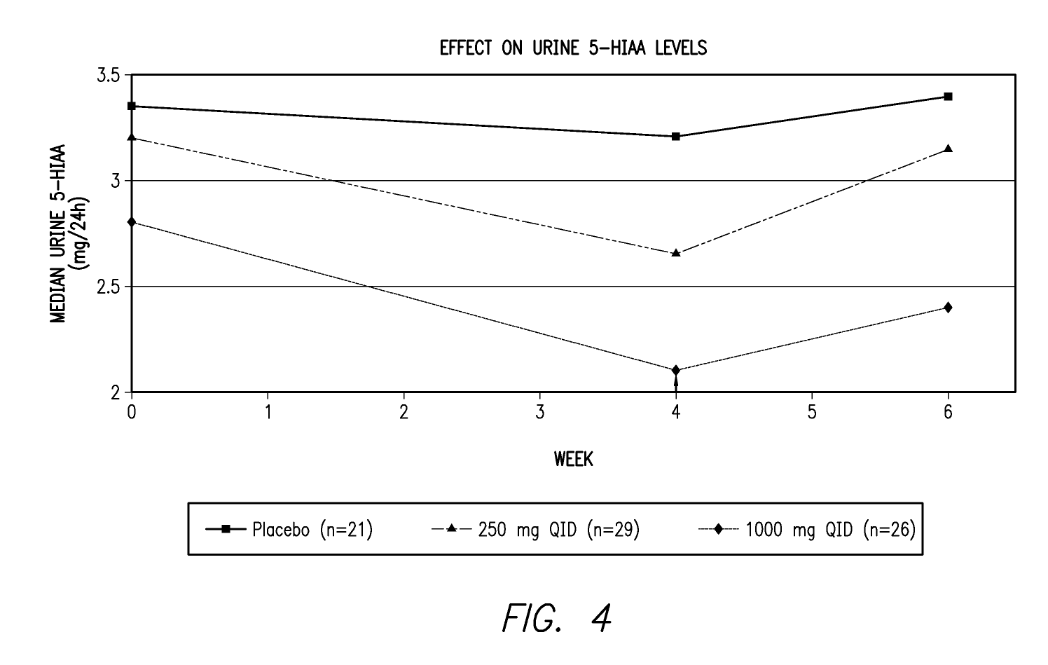

Data obtained from a phase 1b multi-dose study of (S)-2-amino-3-(4-(2-amino-6-((R)-2,2,2-trifluoro-1-(3′-methoxybiphenyl-4-ypethoxy)pyrimidin-4-yl)phenyl)propanoic acid in healthy normal volunteers is shown in FIG. 1. FIGS. 2-4 show data obtained from the phase 2a study in IBS patients.

### 6.2. 5-HIAA Plasma Assay

Quantitative analysis of 5-HIAA in plasma is determined using liquid chromatography mass spectrometry (LC-MS). This method is designed to determine the absolute concentration of 5-HIAA in plasma in a concentration range spanning 0.5 ng/mL to 10,000 ng/mL. This is achieved by spiking 5-HIAA into samples in which the concentration is being determined at eight different levels. The integrated signal of each standard is fit to a calibration curve against which the unknown concentrations are determined. If it is necessary to analyze lower or higher concentration, this curve range can be extended with appropriate dilutions that span the desired concentration range. Where appropriate, an internal standard will be used. If an internal standard is used, the calibration curve will be determined by fitting the area ratios of analyte signal to the internal standard to the standard levels.

Preparation of the calibration standards is accomplished by spiking in 2 μL of the prepared spike-ins into 98 μL of the appropriate matrix. The extraction procedure is a protein precipitation with internal working standard solution, the scheme for which is: 1) Include a control (blank matrix spiked with IS) with each calibration curve; 2) Aliquot 100.0 μL of control matrix (liquid), calibration standards, or study sample (as appropriate) to 1.5 mL polypropylene micro-centrifuge tubes; 3) To control, calibration, and study samples, add 500 μL of internal standard working solution; 4) Vortex-mix all tubes for 2 minutes. Centrifuge at 14000 rpm for 10 minutes; 5) Remove 500 μL of supernatant and place in a 1.5 mL micro-centrifuge tube and dry samples in a Speed-Vac; 6) Reconstitute sample in aqueous mobile phase; 7) Transfer 50 μL aliquots to a 96 well plate; and 8) Inject 10 μLonto the LC/MS/MS system for analysis.

A blank plasma sample is necessary to correct for endogenous levels of 5-HIAA found in plasma. The chromatography method used for this analysis is presented below, although this can be modified as necessary.

Chromatographic Conditions

| Column | Phenomenex Luna Phenyl-Hexyl 50 × 2 mm 3 micron | | |
|---|---|---|---|
| | P/N: 00B-4256-B0 | | |
| Mobile Phase | A: 10 mM Ammonium Acetate/Water | | |
| | B: Acetonitrile | | |
| Flow Rate | 0.20 mL/min. | | |
| Injection Volume | 10 μL with a 10 μL loop | | |
| Column | Room Temp | | |
| Temperature | | | |
| | Time (min) | A (%) | B (%) |
| Gradient | 0 | 99 | 1 |
| | 1.0 | 90 | 10 |
| | 1.1 | 10 | 90 |
| | 1.6 | 10 | 90 |
| | 1.61 | 99 | 1 |
| | 2.5 | 99 | 1 |

| | |
|---|---|
| Acquisition Time | 2.5 minutes |

General conditions for mass spectrometry are given in the table below.

Mass Spectrometric Conditions

| | |
|---|---|
| Instrument | TSQ quantum |
| Ionization Mode | Electrospray (ESI) |
| Polarity | Negative |
| Scan Function | Multiple Reaction Monitoring (MRM) or Selective |
| | Ion Monitoring (SIM) |
| Parameters | The optimization parameters will be chosen for |
| | each analyte. |
| Software | Xcalibur 2.0 For TSQ |

The parent ion and/or product ion, ionization technique, and source parameters will be chosen based on sensitivity and lack of interference near the retention time of the analyte. 5-HIAA ionizes in negative ion ESI mode. It presents a parent ion of 190.0 m/z and a product ion of 144.0 m/z. The samples should be analyzed in the following order: matrix blank, standards, unknown samples and QC samples. Solvent blank samples can be inserted between these sample types.

### 6.3. Effects of (S)-2-amino-3-(4-(2-amino-6-((R)-1-(4-chloro-2-(3-methyl-1H-pyrazol-1-yl)phenyl)-2.2.2-trifluoroethoxy)pyrimidin-4-yl)phenyl)propanoic acid on 5-HIAA Levels

A Phase 1 human clinical trial was conducted as a randomized placebo-controlled trial in healthy volunteers. In the multiple ascending dose study, doses of 250 mg, 500 mg, and 750 mg of (S)-2-amino-3-(4-(2-amino-6-((R)-1-(4-chloro-2-(3-methyl-1H- pyrazol-1-yl)phenyl)-2,2,2-trifluoroethoxy)pyrimidin-4-yl)phenyl)propanoic acid were given three times daily as well as a 1,000 mg twice daily dose were administered over the course of 14 days. Dose groups consisted of eight healthy volunteers with six receiving active compound and two receiving placebo. Study endpoints included safety and tolerability together with pharmacokinetics and pharmacodynamics parameters consisting of measures of both serum and urinary 5-HIAA. In one part of the study, single doses of 250, 500, 1000, 2000, 3000, and 4000 mg were administered to six volunteers, with two volunteers administered placebo. In another part, multiple ascending doses of 250 mg TID, 500 mg TID, 1000 mg BID, and 750 mg TID were administered (six compound, two placebo). Doses were administered fasted (after an overnight fast and 30 minutes prior to the meal).

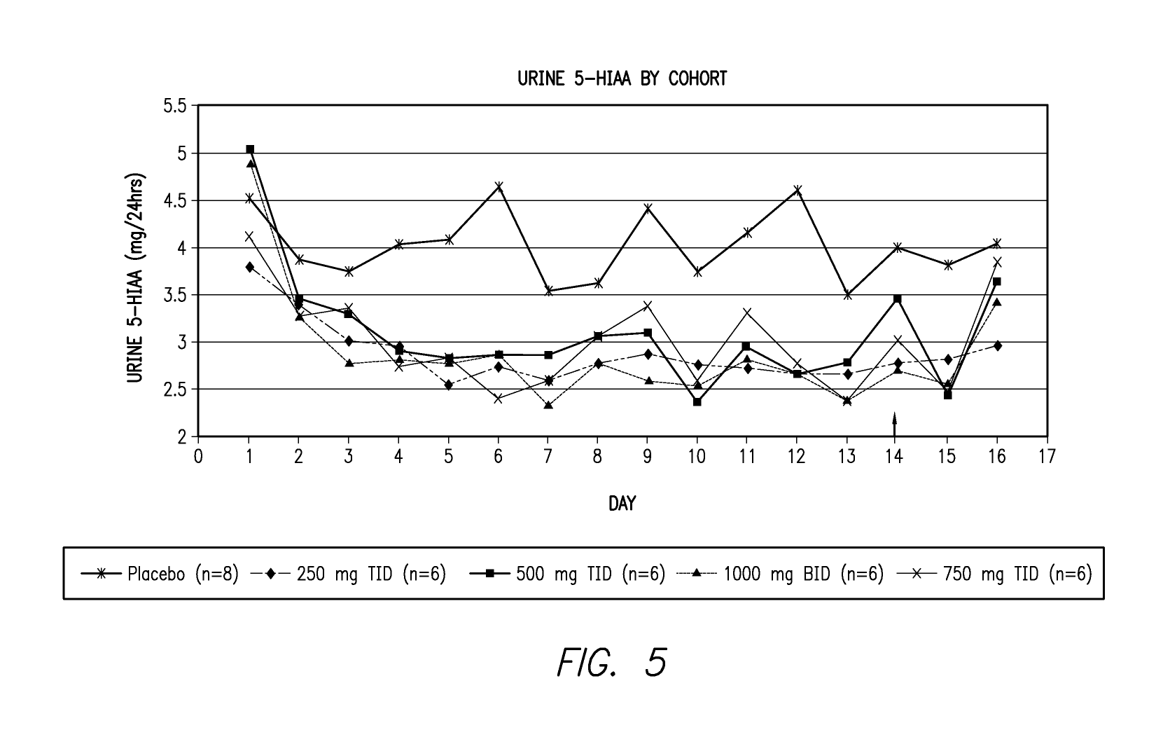

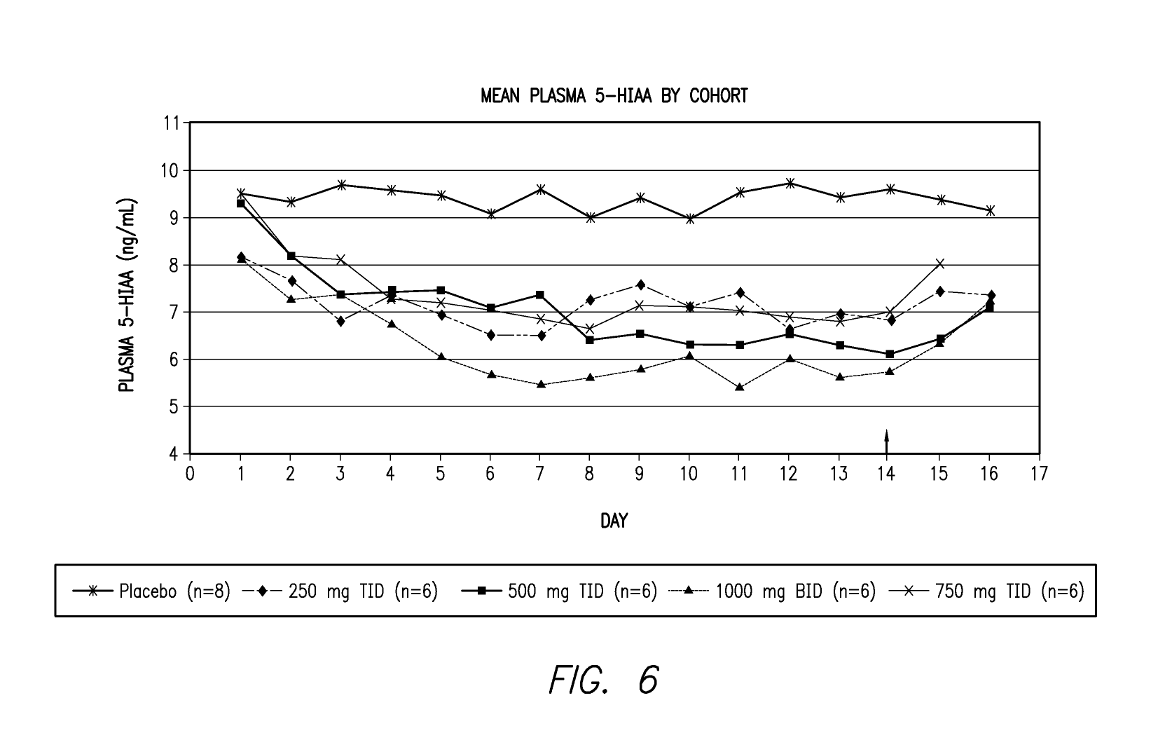

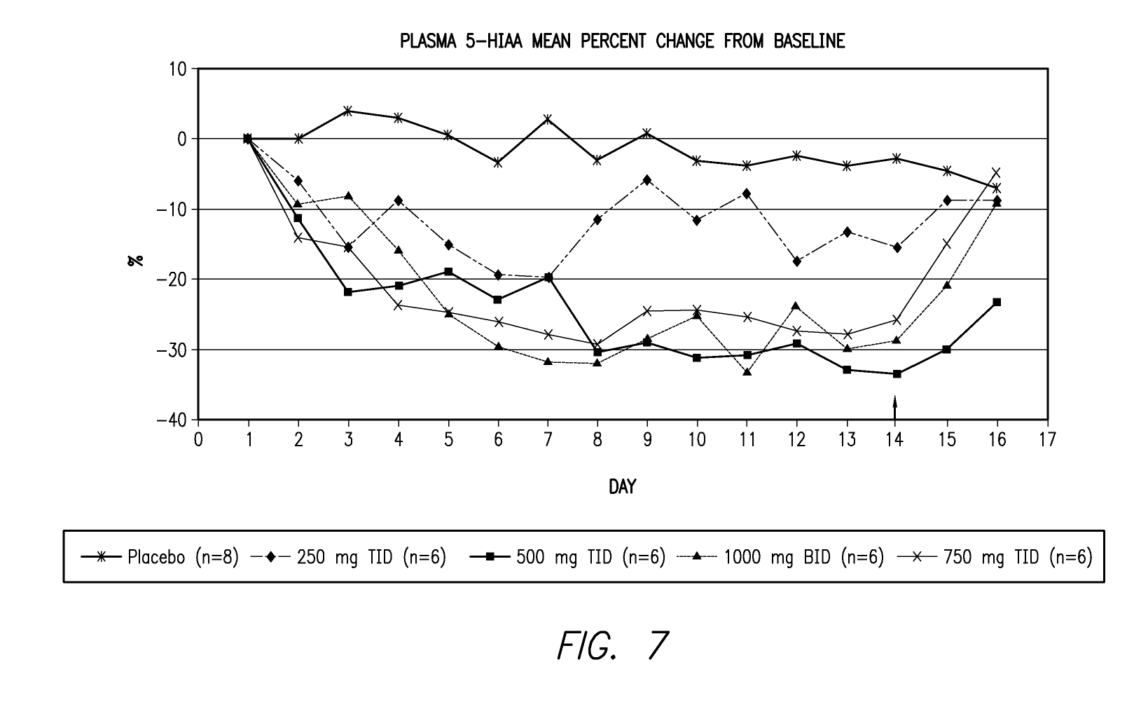

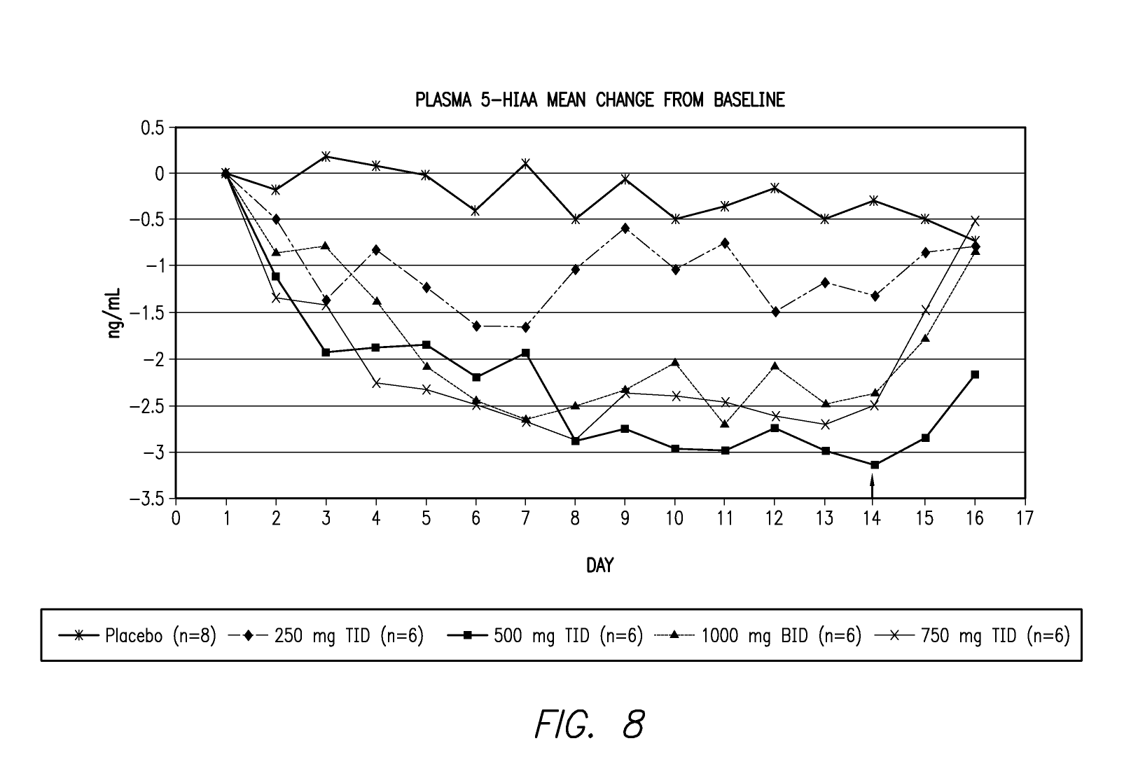

FIGS. 5 and 6 show the effect of the multiple ascending doses of the compound on urinary and plasma mean 5-HIAA levels. The last dose was administered on day 14, which is indicated by an arrow in the figures. Plasma was collected prior to the morning dose. FIGS. 7 shows the mean percent change from baseline plasma 5-HIAA levels, and FIG. 8 shows the mean change from baseline plasma 5-HIAA levels.

All publications (e.g., patents and patent applications) cited above are incorporated herein by reference in their entireties.

1-11. (canceled)

12. A method of treating or managing irritable bowel syndrome (IBS), which comprises administering toe patient suffering from IBS an amount of a TPH inhibitor sufficient to lower the patient peripheral 5-HIAA level by greater than about 10, 20, 30 or 40 percent compared to baseline.

13. The method of claim 12, wherein the 5-HIAA level is determined by measuring plasma 5-HIAA.

14-17. (canceled)

19. A method of estimating the likelihood that a patient suffering from IBS will be responsive to TPH inhibitor therapy, which comprises:
measuring the amount of 5-HIAA in the blood (c.g., plasma) or urine of the patient; and
correlating that amount with a TPH inhibitor responder rate, wherein the TPH inhibitor responder rate is a predicted likelihood of relief from IBS symptoms for patients having a baseline 5-HIAA level that falls within a predetermined range.

20. The method of claim 19, wherein the TPH inhibitor responder rate for the predetermined range is the percent of IBS patients who previously reported experiencing relief from IBS symptoms and who had baseline 5-HIAA levels that fell within the predetermined range.

21. The method of claim 19, wherein the TPH inhibitor responder rate for the predetermined range is the percent of IBS patients who previously reported a decrease in mean stool consistency and who had baseline 5-HIAA levels that fell within the predetermined range.

22-26. (canceled)

27. A kit for determining whether a patient suffering from IBS will be responsive to TPH inhibitor therapy, which comprises:
a device for measuring 5-HIAA concentrations; and
information that correlates or provides instructions as to how to correlate measurements obtained using the device with a TPH inhibitor responder rate, wherein the TPH inhibitor responder rate is a predicted likelihood of relief from IBS symptoms for patients having a baseline 5-HIAA level that falls within a predetermined range.

28. The kit of claim 27, which further comprises a questionnaire that can be used to determine whether the patient has IBS-D or IBS-A.

29. The kit of claim 27, wherein the device utilizes an ELIZA.

30. The kit of claim 27, wherein the TPH inhibitor responder rate for the predetermined range of 5-HIAA levels is the percent of IBS patients who previously reported experiencing relief from IBS symptoms and who had baseline 5-HIAA levels that fell within that predetermined range.

31. The kit of claim 27, wherein the TPH inhibitor responder rate for the predetermined range of 5-HIAA levels is the percent of IBS patients who previously reported a decrease in mean stool consistency and who had baseline 5-HIAA levels that fell within that predetermined range.
